SEQUENCE LISTING (21 sequences)

SEQ ID NO: 1 - DNA, 20 bp, Artificial
  misc_feature: PCR primer
ccttcctcctgtgcctgatg

SEQ ID NO: 2 - DNA, 22 bp, Artificial
  misc_feature: PCR primer
acaatctcatttgaatcaggaa

SEQ ID NO: 3 - DNA, 22 bp, Artificial
  misc_feature: PCR probe
tgcccgactcccttgggtgtca

SEQ ID NO: 4 - DNA, 18 bp, Artificial
  misc_feature: PCR primer
acggcgctgtcatcgatt

SEQ ID NO: 5 - DNA, 20 bp, Artificial
  misc_feature: PCR primer
ggcattcttcacctgctcca

SEQ ID NO: 6 - DNA, 27 bp, Artificial
  misc_feature: PCR probe
cttccctgtgaaaacaagagcaaggcc

SEQ ID NO: 7 - DNA, 20 bp, Artificial
  misc_feature: PCR primer
gcccaggcagtcagatcatc

SEQ ID NO: 8 - DNA, 21 bp, Artificial
  misc_feature: PCR primer
gggtttgctacaacatgggct

SEQ ID NO: 9 - DNA, 24 bp, Artificial
  misc_feature: PCR probe
ctcgaaccccgagtgacaagcctg

SEQ ID NO: 10 - DNA, 19 bp, Artificial
  misc_feature: PCR primer
caccggaacgacatggaga

SEQ ID NO: 11 - DNA, 20 bp, Artificial
  misc_feature: PCR primer
tccaggcgacaaaagggtta

SEQ ID NO: 12 - DNA, 27 bp, Artificial
  misc_feature: PCR probe
tgggcttcgtctactcctttctgggtc

SEQ ID NO: 13 - DNA, 20 bp, Artificial
  misc_feature: PCR primer
gcctggcctccagaaagacc

SEQ ID NO: 14 - DNA, 29 bp, Artificial
  misc_feature: PCR primer
acctggtacatcttcaagtcttcataaat

SEQ ID NO: 15 - DNA, 26 bp, Artificial
  misc_feature: PCR probe
cttttatgatggccctgtgccttagt

SEQ ID NO: 16 - DNA, 21 bp, Artificial
  misc_feature: PCR primer
gccaggagttgtgagtttcca

SEQ ID NO: 17 - DNA, 19 bp, Artificial
  misc_feature: PCR primer
tgcaaggcccttcatgatg

SEQ ID NO: 18 - DNA, 24 bp, Artificial
  misc_feature: PCR probe
tctgaccacttctctgcctgccca

SEQ ID NO: 19 - DNA, 20 bp, Artificial
  misc_feature: PCR primer
gttccccatatccagtgtgg

SEQ ID NO: 20 - DNA, 20 bp, Artificial
  misc_feature: PCR primer
tcctttgcaagcagaactga

SEQ ID NO: 21 - DNA, 8 bp, Artificial
  misc_feature: PCR probe
tggctgtg

(19) United States
(12) Patent Application Publication
(10) Pub. No.: US 2011/0033843 A1
(43) Pub. Date: Feb. 10, 2011

(54) METHOD FOR IN VITRO TESTING OF COMPOUNDS FOR ASSESSING THERAPEUTIC VALUE IN THE TREATMENT OF MULTIPLE SCLEROSIS AND OTHER DISEASES WHEREIN FOAMY CELLS ARE INVOLVED IN THE DISEASE ETIOLOGY

(76) Inventors: Leonie Alexander Boven, Rotterdam (NL); Jon D. Laman, Amsterdam (NL); Edward E.S. Nieuwenhuis, Rotterdam (NL)

Correspondence Address: TRASKBRITT, P.C., P.O. BOX 2550, SALT LAKE CITY, UT, 84110, US

(21) Appl. No.: 12/085,426

(22) PCT Filed: Nov. 21, 2006
(86) PCT No.: PCT/EP2006/011143

§ 371 Date: May 23, 2008

Related U.S. Application Data
(60) Provisional application No. 60/739,550, filed on Nov. 23, 2005.
(60) Provisional application No. 60/800,883, filed on May 16, 2006.

(51) Int. Cl. C12Q 1/68; G01N 33/566

(52) U.S. Cl. 435/6; 435/7.21

(57) ABSTRACT

The invention provides a method for assessing or determining activity of a test Compound on modulation of gene product levels comprising culturing cells, contacting at least one of the cultured cells with a lipid-rich fraction, contacting at least one of the cultured cells with the test Compound, determining the presence of a gene product of at least one cell of the cultured cells and, optionally, determining the presence of the gene product of at least one cultured cell not contacted with the test Compound. To assess human conditions most fully, it is preferred that the cell is of human origin, for example, a peripheral blood monocyte taken from a healthy donor.

### TECHNICAL FIELD

The invention relates to the field of multiple sclerosis (MS) and to experimental models that are useful to test pharmaceutical compounds.

### BACKGROUND

Multiple sclerosis is a chronic inflammatory autoimmune disease of the central nervous system (CNS) and is characterized by the presence of demyelinated areas throughout the CNS. Various mechanisms leading to demyelination and axonal suffering have been implicated and the production of toxic inflammatory mediators by infiltrating and resident CNS macrophages is believed to play a pivotal role. MS is thought to be caused by a combined cellular and humoral autoimmune attack on myelin sheaths and possibly axons. Several facts have contributed to the concept that MS is an autoimmune disease, such as the association with various regulatory genes of the immune response, the presence of oligoclonal immunoglobulin species in CSF pointing to intrathecal expansion of specific B-cell clones, and the immunopathology of the lesions. Further support comes from the immunopathological similarity of MS with the autoimmune animal model EAE (experimental autoimmune/allergic encephalomyelitis) in rodents and primates, which, considering that no in vitro models exist, is the only experimental model existing so far that may be used to test scientific hypotheses on the critical pathogenetic mechanisms and for the development of more effective therapies. However, the substantial dissimilarities between MS and EAE models have among others raised doubts about the autoimmune origin of MS. Notably, many of the EAE models present as a rapidly progressing monophasic disease with clinical and pathological findings that are more reminiscent of acute disseminated encephalomyelitis than chronic and relapsing MS. Although exceptions do exist, such as the elegant EAE model in Biozzi/ABH mice immunized with spinal-cord homogenate and a non-human-primate model for chronic MS in common marmosets that approximate the human disease better, currently no existing experimental model bridges the considerable gap between EAE models and MS.

Different subsets of myeloid cells are considered to have distinct roles in the development of MS. These distinct and specialized roles of myeloid cells depend on their origin and, importantly, their location. As such, perivascular cells appear to be optimally positioned for the modulation of infiltrating T-cell activity whereas parenchymal myeloid cells may have a more prominent role in mechanisms involved in myelin breakdown and axonal suffering.

The plasticity and functional polarization of macrophages have received renewed attention in light of novel key properties of different forms of macrophages. Two extremes of a continuum have been identified for macrophages, being M1, or classically activated macrophages, and M2, or alternatively activated macrophages. The M1 phenotype is typically induced in vitro by IFN-gamma, TNF-alpha or LPS, whereas the M2 phenotype can be induced by IL-10, IL-4 or by the lipid mediator PGE₂, which is a strong inhibitor of pro-inflammatory immune responses. M1 macrophages are characterized by a high production of pro-inflammatory mediators and are involved in Th1 cell responses and killing of micro-organisms and tumor cells. In contrast, M2 macrophages are associated with Th2 responses, scavenging of debris, promotion of tissue remodeling and repair and expression of anti-inflammatory molecules, including IL-1ra (IL-1 receptor antagonist) and CCL18. CCL18 in particular is a specific marker for human alternatively activated macrophages and is involved in immune suppression.

Demyelinating MS lesions are characterized by the presence of foamy macrophages, a characteristic subset of myeloid cells, which acquire their distinctive morphology by ingestion and accumulation of vast amounts of myelin-derived lipids. Foamy macrophages originate from both resident microglia and infiltrating monocytes, and about 30 to 80% of foamy macrophages in demyelinating lesions are blood-derived. Besides their apparent role in scavenging myelin, it is still poorly understood if and how foamy macrophages may affect the local inflammatory process. Since MS lesions are self-limiting and do not expand indefinitely it is likely that local mechanisms restrict CNS inflammation and may also promote tissue repair. It is however up to now not clear how these local mechanisms may function.

### DISCLOSURE OF THE INVENTION

The invention provides a method for assessing or determining activity of a test compound on modulation of gene product levels comprising culturing (preferably myeloid) cells, contacting at least one of the cultured cells with a lipid-rich fraction, contacting at least one of the cultured cells with the test compound, determining the presence of a gene product of at least one cell of the cultured cells, and optionally determining the presence of the gene product of at least one cultured cell not contacted with the test compound. To assess human conditions most fully, it is preferred that the cell is of human origin, for example, a peripheral blood monocyte or granulocyte taken from a healthy donor. Also, the invention provides a method for assessing or determining activity of a test compound on modulation of gene product levels in more specific circumstances of disease, it is then preferred the myeloid cell has been derived from a subject thought to be suffering from a disease. Use of a method according to the invention would then allow for individualized medicine; test results indicating that a specific test compound has specific benefits for the subject may then be used for treatment of the subject against the disease.

Specific disease conditions that can be studied by a method according to the invention are those diseases wherein foamy cells are considered involved in the etiology of the disease, such as is the case with multiple sclerosis or atherosclerosis. This disease definition includes disease in which cells with a foam cell morphology have a modulatory function in either disease initiation, progress and aggravation, or in disease reduction, amelioration, inflammation control, tissue integrity-tissue homeostasis: multiple sclerosis (MS), atherosclerosis (in the broad sense of the word, so including angina pectoris, myocardial infarction, stroke, vulnerable plaque syndrome), diabetes, lung disease in general (including chronic inflammation, asthma, emphysema, viral, bacterial, fungal and parasitic infection, as well as genetic aberrations such as cystic fibrosis, pulmonary alveolar proteinosis, inflammatory bowel disease (IBD, including morbus Crohn and colitis ulcerosa), genetic deficiencies affecting lipid storage (e.g., Gaucher disease) and Hodgkin's disease. The invention also provides a method to test efficacy or mechanism of action of candidate drugs or combinations of drugs for the disease of interest. It is herein also provided to use a method according to the invention as application of test system for individualized medicine; i.e., in diagnosis-prognosis studies, in assessment of individuals risk to develop disease related to foam cell (dys)function, wherein we here provide a method to predict disease risk, in conjunction with known risk factors (e.g., age, weight, gender, smoking for atherosclerosis). We can also now assess individual patient for their response to drug treatment in vitro, or ex vivo, and thus select the right patient-drug combination, and/or test combinations of drugs, and/or assess responsiveness of patient to establish dose to be used in treatment, e.g., by culturing relevant cell type(s) taken from peripheral blood of the individual in the presence of the appropriate source and form of foam cell inducing compound (i.e., myelin, oxLDL). By assessing drug response by titrating in the drug both during development of foam cells over a one to three day period, or when foam cells have been established will provide guidance in drug-selection.

Myeloid cells that can advantageously be used are derived from myeloid cell lines such as U937 (human): ATCC CRL-1593.2; THP-1 (human): ATCC TIB-202; RAW (mouse): ATCC TIB-71. In vitro foam cells are also cells having acquired a large bloated irregular morphology with multiple vesicles due to (excessive) lipid, glycolipid or sugar uptake, and/or increased intracellular production, and/or reduced degradation-catabolism of such compounds. As to the lipid-rich fraction to be used in a method according to the invention, it is preferred that the lipid-rich fraction comprises one or more compounds selected from the group of phospholipids cholesterol, sphingolipids, glycolipids, ceramides, such as can be found in myelin, for example, in soluble form, or in particulate form being multiple membrane windings of oligodendrocyte extensions forming the multilamellar myelin sheath, or in particulate form being apoptotic cell bodies from oligodendrocytes, axons, neuron somata, astrocytes, microglia, and/or infiltrating white blood cells.

As to the detection of gene products involved, the invention, for example, provides a method for assessing or determining activity of a test compound on modulation of gene product levels comprising culturing myeloid cells, contacting at least one of the cultured cells with a lipid-rich fraction, contacting at least one of the cultured cells with the test compound, determining the presence of a gene product of at least one cell of the cultured cells, wherein the gene product is a proteinaceous substance such as a peptide, polypeptide or protein, having or not having been modified with post-translational modifications. Also, the invention provides a method wherein the gene product is a cytokine or chemokine. Gene products such as (m)RNA or specific parts thereof may also be detected, thereby allowing for identifying transcriptional activity in the cell that may or may not be influenced by the test compound under study. Again, a useful example of RNA testing comprises testing for RNA that at least partially encodes a cytokine or chemokine. Another useful example of RNA testing comprises testing for RNA that at least partially encodes a liver X receptor (LXR) or LXR-induced genes. Yet another useful example of RNA testing comprises testing for RNA that at least partially encodes an adenosine receptor or adenosine receptor-induced genes. Of course, proteins or peptides encoded by the RNA can also be tested. Cell types preferably used in the invention are cells which have means to take up compounds from the environment by cell biological processes including micropinocytosis, macropinocytosis, phagocytosis are useful. In principle, under the appropriate tissue or culture conditions, many different cell types potentially acquire foam cell morphology and may be used. However, various cell types optimally equipped for phagocytosis are prime candidates for transformation into foam cells. These include for leukocytes: cell types from the myeloid and granulocyte series (monocytes-macrophages, neutrophils, eosinophils and basophils). Also, dendritic cells (DC) are a leukocyte subset useful to the method. The origin of DC is disputed to some extent, with extensive debate on myeloid versus lymphoid DC, but clearly DC precursors are present in the circulation. DC can, for example, be generated in vitro from human monocytes and from bone marrow (mouse and human) in appropriate cytokine mixtures. Multiple sclerosis-associated cell types likely to turn into foam cells are microglia (brain macrophages) and infiltrating macrophages and DC. Also pericytes might be candidates. Neutrophils may be important in early lesions, and have phagocytic activity. Also rat, mouse, marmoset and rhesus monkeys cells (for which EAE-MS models have been established) develop into foam cells upon myelin exposure. For studying atherosclerotic disease it is useful to use macrophages or human monocytes to transform into foam cells mimicking those in the plaque, notably by uptake of oxLDL by means of scavenging receptors (SR-A, SR-B, CD36) and TLR (e.g., TLR4, TLR2). Other cell types to be considered are neutrophils, smooth muscle cells (SMC), fibroblasts and myofibroblasts. Prime candidates to study lung disease are alveolar macrophages and macrophages/phagocytes in the connective tissue of the lung. Other cell types to be considered are neutrophils, smooth muscle cells (SMC), fibroblasts, myofibroblasts, and type I and type II pneumocytes.

In particular, the invention provides a method to practice an in vitro model of MS. This method, for example, comprises a step of culturing a (preferably myeloid) cell or cells, preferably of human origin, such as a human blood monocyte obtained from a donor, if required differentiating the monocyte into other cell types, such as macrophages and dendritic cells, and a step of contacting the cultured cell with a lipid-rich fraction, preferably a phospholipid rich fraction, preferably with a myelin-rich fraction, and a third step of culturing the cell in the presence of the lipid-rich fraction until the cell or at least 10% of the cells, preferably at least 20%, more preferably at least 30%, more preferably at least 40%, more preferably at least 50%, more preferably at least 60%, more preferably at least 70%, more preferably at least 80%, most preferably at least 90%, have developed a foamy characteristic because of the ingestion of the lipid-rich fraction, as can be observed by light microscope or as can be determined by staining the cell or cells for the intracellular presence of lipid-rich fractions, as for example, can be done by staining the cell or cells or a fraction thereof with a stain for the detection of neutral lipids, such as by staining with oil red 0 histochemistry (ORO) or by fluorescent labeling of lipids with DiI and subsequent detection of ingested fluorescent lipids. Letting foam cells stand in culture for a too long period without feeding a lipid-rich fraction will make them return to a non-foamy character; it then suffices to refeed them a lipid-rich fraction to induce the foamy morphology again. In one embodiment of the invention, human myeloid cells obtained from healthy donors are fed with 10 to 200, preferably with about 50 microg/ml human myelin, for example, purified from postmortem brain. In another embodiment of the invention, mouse primary macrophages obtained from healthy mice are fed with 10 to 200, preferably with about 50 microg/ml human or mouse myelin. In another embodiment of the invention, marmoset myeloid cells obtained from healthy donors are fed with 10 to 200, preferably with about 50 microg/ml marmoset myelin. In another embodiment of the invention, human primary macrophages obtained from healthy donors are fed with 10 to 200, preferably with about 50 microg/ml phospholipid. Although small individual changes in kinetics between individual donors may be observed, myeloid cells acquire a foamy morphology between 24 and 48 hours and contain a markedly increased number and size of lipid droplets in comparison to control cells (i.e., not fed with lipid) as, for example, demonstrated by ORO staining. Lipid droplets in cells not exposed to myelin likely derive from lipid in the culture medium and/or apoptotic other macrophages in the culture. Primary macrophages may be used but also myeloid or monocyte-like cells or cell lines such as U937 (human): ATCC CRL-1593.2; THP-1 (human): ATCC TIB-202; RAW (mouse): ATCC TIB-71, or specific monocyte-like cells such as rodent, marmoset or human myeloid dendritic cells (mDC) or microglial cells can develop the foamy characteristics when fed lipid-rich fraction and are advantageously used in a method as provided herein.

We hypothesized that foamy macrophages in MS brain are anti-inflammatory M2-type macrophages as generated under laboratory conditions. We then hypothesized that foamy macrophages actively contribute to the resolution of brain inflammation. Our findings reveal an important and previously overlooked anti-inflammatory role for foamy macrophages in MS lesions. The invention provides the insight that multiple sclerosis (MS) lesion activity concurs with the extent of inflammation, demyelination and axonal suffering, in short, with the balance between local pro- and anti-inflammatory activities. Pro-inflammatory myeloid cells contribute to lesion development, but the self-limiting nature of lesions now is explained as earlier unidentified anti-inflammatory mechanisms. We show herein that lipid ingestion, and in particular myelin ingestion by myeloid cells induces a foamy appearance and confers anti-inflammatory function. We show that myelin-containing foam cells in MS lesions consistently express a series of anti-inflammatory molecules while mainly lacking pro-inflammatory cytokines. Unique location-dependent cytokine and membrane receptor expression profiles allow for functional specialization allowing for differential responses to micro-environmental cues. The invention therewith provides a novel, and advantageously an essentially human in vitro model of MS using foamy macrophages wherein it functionally is confirmed that in human macrophages myelin ingestion induces an anti-inflammatory program, to which program the effects of test compounds can be evaluated. The invention also provides novel insights into the mechanisms of lesion control and opens new roads to therapeutic intervention at the exact site where it most counts in MS, the recurrent inflammatory lesion in the brain.

### DESCRIPTION OF THE DRAWINGS

FIG. 1 Microlocation-dependent expression profiles of surface and intercellular molecules by foamy macrophages in MS lesions. The presence of CNS proteins, molecules involved in antigen recognition and presentation, as well as anti- and pro-inflammatory molecules was analyzed for foamy macrophages at different sites within the lesion, i.e., in the outer rim, the inner rim, its lesion center and in perivascular spaces. Quantification was based on frequency of positive foamy macrophages and staining intensity and was performed on two to three lesions from four different MS patients. The gradient between lesion center and the perivascular space reflects increasing or decreasing staining frequency and/or intensity towards the perivascular compartment.

FIG. 2 Foamy macrophages in vitro are immunosuppressive. (a) Foamy macrophages were generated using myelin preparations derived from brain tissue of three control individuals and three MS patients. After addition of 1 ng/ml LPS for 24 hours, cytokine levels in the supernatants were determined. LPS-induced IL-12p40 and IL-10 production was dose-dependently inhibited by myelin and is shown as the percentage of production by untreated LPS-stimulated macrophages. Control patient-derived myelin, filled squares; MS patient-derived myelin, open squares. (b) Foamy macrophages were incubated with myelin for 24 hours and subsequently stimulated with 1 ng/ml LPS for two hours where indicated. LPS-induced IL-12p35 and TNF-alpha mRNA levels were significantly inhibited in foamy macrophages compared to control macrophages. LPS-induced IL-10 and COX-2 were not affected. PGES and CCL18 mRNA expression was not induced significantly by LPS and was 0 increased by myelin. *, P<0.05 compared with LPS-treated macrophages. (c) Over time, foamy macrophages showed a decreased, but not significant, IL-12p35 mRNA expression. In addition there was a transient increase in PGES and a sustained increase in CCL18 mRNA expression. *, P<0.05 compared with untreated control macrophages. (d) This was paralleled on protein level as seven days after myelin addition, foamy macrophages still showed significantly increased CCL18 production. *, P<0.05 compared with untreated control macrophages. (e) Seven days after myelin ingestion, foamy macrophages showed a complete inhibition of LPS-induced IL-10 and IL-12p40 production and a three-fold induction of CCL18 production. *, P<0.05 compared with LPS-treated macrophages. All data shown are representative for at least two independent experiments using different blood donors. Results are expressed as mean±s.d.

FIG. 3. Expression of LXRalpha mRNA is increased by myelin-laden macrophages of three different healthy donors. A) Primary human macrophages were cultured in the absence or presence of 25 microgram/ml myelin for 48 hours and subsequently stimulated with 1 ng/ml LPS where indicated. Myelin-induced LXR alpha expression three-fold and LPS did not have an additional effect. B) Myelin was added for the indicated time points and induced LXRalpha expression up to five-fold after five days. Addition of LPS for six hours further induced LXRalpha expression. C) Myelin was added for the indicated time points and induced LXRalpha expression up to five-fold after five days. Addition of LPS did not affect LXRalpha expression.

FIG. 4. Expression of ABCA1 mRNA is increased by myelin-laden macrophages of three different donors. A) Primary human macrophages were cultured in the absence or presence of 25 microgram/ml myelin for 48 hours and subsequently stimulated with 1 ng/ml LPS where indicated. Myelin-induced ABCA1 expression three-fold and LPS did not have an additional effect. B) Myelin was added for the indicated time points and induced ABCA1 expression up to eight-fold after five days. Addition of LPS for six hours further induced ABCA1 expression. C) Myelin was added for the indicated time points and induced ABCA1 expression up to 2.5-fold after two days. Addition of LPS for six hours further induced ABCA1 expression.

FIG. 5. LXRalpha and ABCA1 mRNA expression are increased in MS brain tissue containing demyelinated lesions. (A) ABCA1 mRNA expression was significantly increased (p=0.003, Kruskal Wallis) in lesional MS brain tissue as compared to control tissues. (B) LXR-alpha mRNA was increased in the group with MS lesions, although not significantly. The right panel shows the individual patients. Tissue of MS patients 5, 9, 10, 11 only consisted of normal appearing white matter. The left panel shows scatter plots of the three patient groups.

FIG. 6. ABCA1 and LXRalpha mRNA levels correlate significantly between individuals (p<0.01, Spearman's).

FIG. 7. Expression of A1 receptor mRNA by myelin-laden macrophages of two healthy donors. A) Primary human macrophages were cultured in the absence or presence of 25 microgram/ml myelin for 48 hours and subsequently stimulated with 1 ng/ml LPS for two or six hours where indicated. Myelin nor LPS had a significant effect on A1 receptor mRNA expression. B) Myelin was added for the indicated time points and induces A1 receptor mRNA expression up to three-fold after five days. Addition of LPS for six hours reduced A1 receptor mRNA expression.

FIG. 8. Expression of A2a receptor mRNA by myelin-laden macrophages of two healthy donors. A) Primary human macrophages were cultured in the absence or presence of 25 microgram/ml myelin for 48 hours and subsequently stimulated with 1 ng/ml LPS for two or six hours where indicated. Myelin increased A2a receptor mRNA expression three-fold. LPS-induced A2a receptor mRNA up to 250-fold in both control and foamy macrophages. B) Myelin was added for the indicated time points and induced A2a receptor mRNA expression up to six-fold after seven days. Addition of LPS further induced A2a receptor mRNA expression till seventy-fold after seven days of myelin.

FIG. 9. Expression of A2b receptor mRNA by myelin-laden macrophages of two healthy donors. A) Primary human macrophages were cultured in the absence or presence of 25 microgram/ml myelin for 48 hours and subsequently stimulated with 1 ng/ml LPS for two or six hours where indicated. Myelin nor LPS affected A2b receptor mRNA expression. B) Myelin was added for the indicated time points and did not affect A2b receptor mRNA expression. Addition of LPS down-regulated A2b receptor mRNA expression in control and foamy macrophages.

FIG. 10. Expression of A3 receptor mRNA by myelin-laden macrophages of two healthy donors. A) Primary human macrophages were cultured in the absence or presence of 25 microgram/ml myelin for 48 hours and subsequently stimulated with 1 ng/ml LPS for two or six hours where indicated. Myelin down-regulated A3 receptor mRNA expression. LPS further down-regulated A3 receptor mRNA expression. B) Myelin was added for the indicated time points and down-regulated A3 receptor mRNA expression. Addition of LPS further down-regulated A3 receptor mRNA expression in control and foamy macrophages.

FIG. 11. A1, A2a, A2b, and A3 receptor mRNA expression in MS brain tissue and non-demented controls. (A-C) mRNA expression of A1, A2a, A2b receptor was not altered in lesional MS brain tissue as compared to MS normal appearing white matter (NAWM) or control tissues. (D) A3 receptor mRNA was significantly increased in the group with MS lesions (p<0.005, Kruskal Wallis). The right panel shows the individual patients. Tissue of MS patients 5, 9, 10, 11 only consisted of normal appearing white matter. The left panel shows scatter plots of the three patient groups.

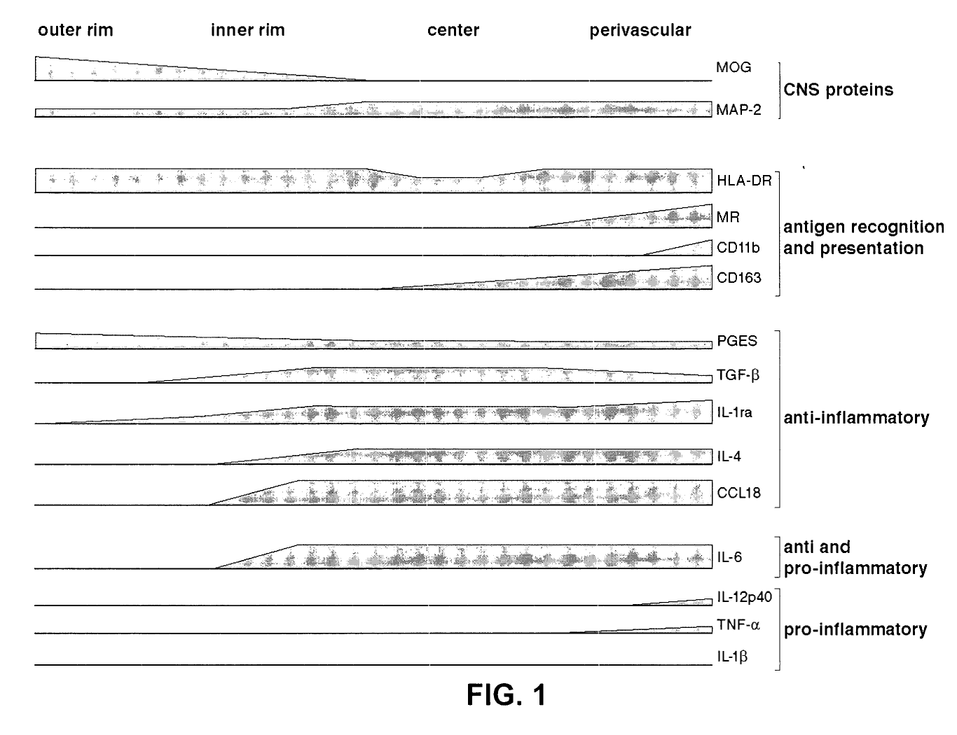

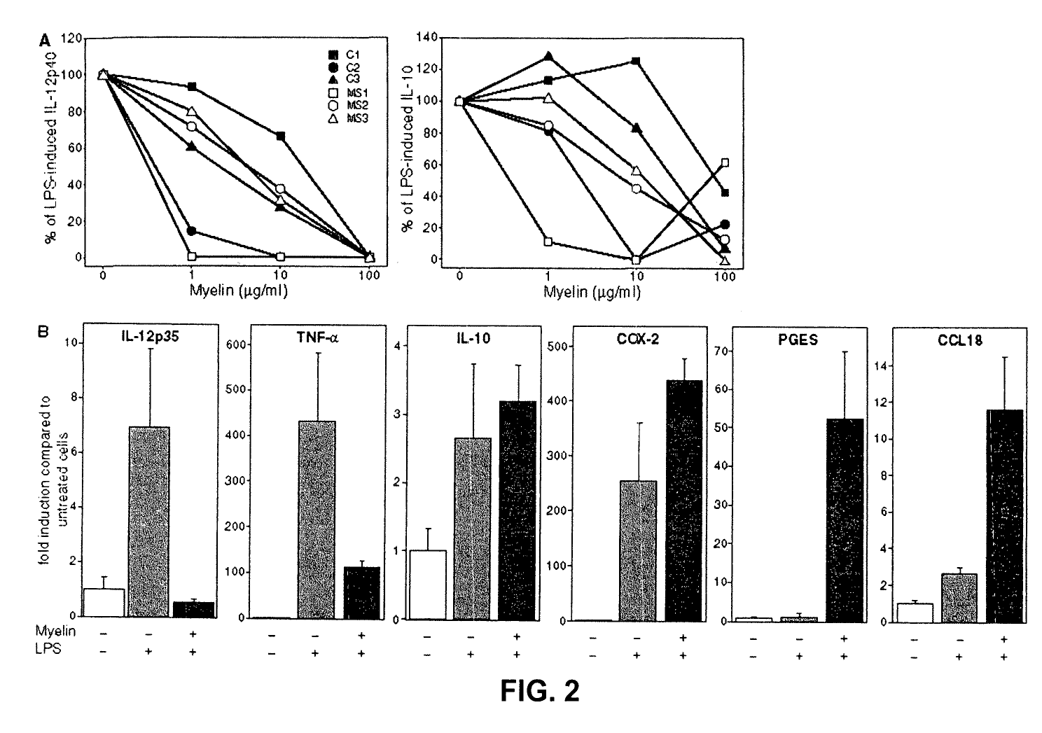

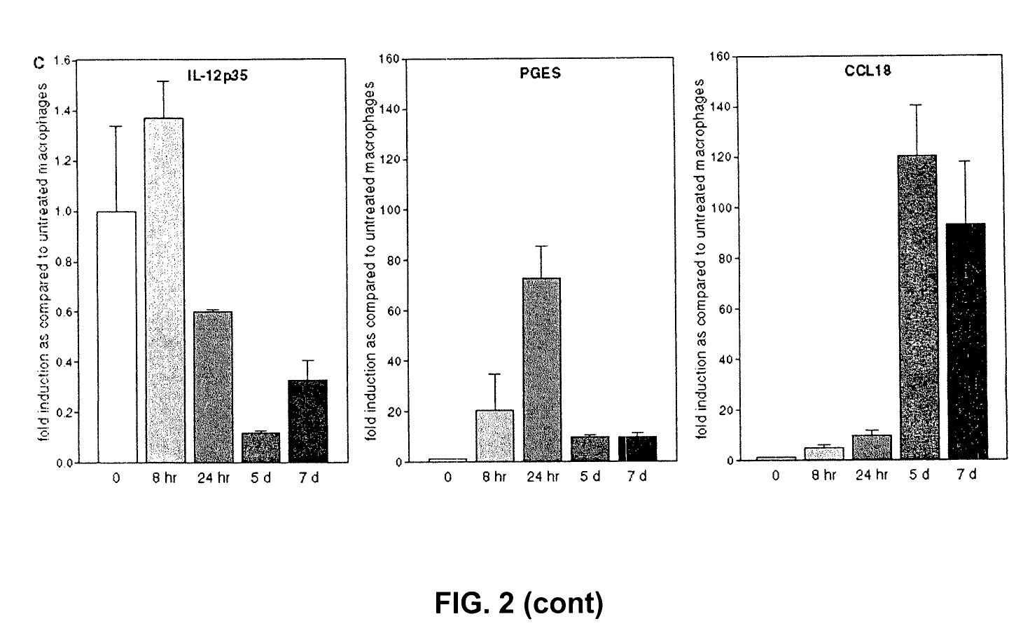

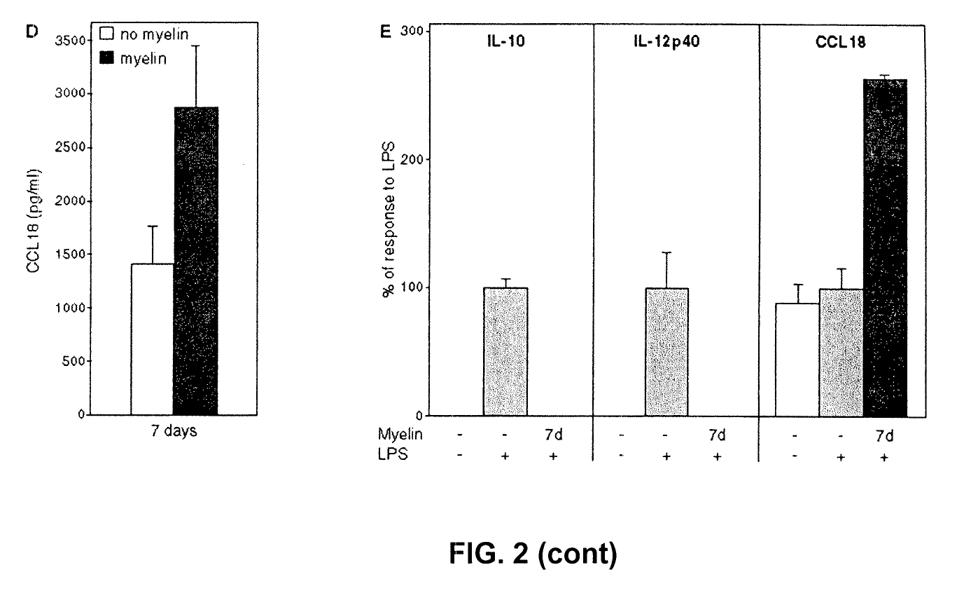

### MODE(S) FOR CARRYING OUT THE INVENTION

Abbreviations used: LPS, lipopolysaccharide; IL, Interleukin; PGE, prostaglandin E; EAE, experimental autoimmune encephalomyelitis; Th, T helper; ATCC, American Type Culture Collection; IL-1ra, receptor antagonist; HLA, human leukocyte antigen; TGF, transforming growth factor; ELISA, enzyme-linked immuno sorbent assay; COX, cyclooxygenase; TNF, tumor necrosis factor; IFN, interferon; MS, Multiple sclerosis; CNS, Central nervous system; NAWM, Normal appearing white matter; MOG, Myelin oligodendrocyte glycoprotein; ORO, Oil red O;

TABLE 1
Markers and antibodies

| Molecule/marker | Function |
|---|---|
| IL-1ra | Anti-inflammatory, Endogenous IL-1 antagonist |
| IL-4 | Anti-inflammatory |
| PGES | Anti-inflammatory |
| TGF-beta | Anti-inflammatory |
| CCL18 | expressed by T/B-cells, DC, macrophages, |
| | chemotactic to naïve T-cells and iDC |
| HLA class II | Antigen presentation to CD4+ T-cells |
| CD163 | Scavenger receptor for haptoglobin-haemoglobin |
| | complexes, anti-inflammatory actions |
| Mannose receptor | Lectin, recognition of micro-organisms |
| CD11b | Forms complement receptor 3 with CD18 |
| IL-1beta | Pro-inflammatory cytokine |
| TNF-alpha | Pro-inflammatory cytokine |
| IL-6 | Pro- and anti-inflammatory actions |
| IL-12 p40/p70 | Pro-inflammatory cytokine |
| MOG | Myelin oligodendrocyte glycoprotein |
| MAP-2 | Neuronal protein |

### Example 1

### Myelin-Laden Macrophages are Anti-Inflammatory Consistent with Foam Cells in Multiple Sclerosis

### Material and Methods: Immunohistochemical Analysis of Postmortem MS Brain Tissue

Human autopsy brain tissue from five MS patients was provided by the Netherlands Brain Bank in Amsterdam. Immunohistochemistry was performed on frozen sections of MS brain tissue to detect expression of (anti-)inflammatory markers and CNS antigens (Table I) as described previously (Hoefakker et al., 1995). In brief, 6 μm frozen sections were cut and thawed on to glass slides. Slides were kept overnight at room temperature in humidified atmosphere. After air-drying, slides were fixed in acetone containing 0.02% (v/v) H₂O₂. Slides were then air-dried for ten minutes, washed with PBS and incubated with optimally diluted primary antibody overnight at 4° C. in humidified atmosphere. Incubations with secondary rabbit anti-mouse-Ig-biotin (Dako) and tertiary horseradish peroxidase (HRP)-labeled avidin-biotin-complex (ABC/HRP: Dako) were performed for one hour at room temperature. HRP activity was revealed by incubation for ten minutes at room temperature with 3-amino-9-ethyl-carbazole (AEC: Sigma), leading to a bright red precipitate. After washing, sections were counterstained with hematoxylin, and embedded with glycerol-gelatin. Omission of primary antibody acted as control staining. Myelin degradation products were detected with oil-red O (ORO), which stains neutral lipids, as previously described (Chayen and Bitensky, 1991). The used antibodies were the anti-inflammatory markers IL-1ra (Biosource), IL-4 (U-Cytech), PGES (Cayman), TGF-beta (Santa cruz), and CCL18 (R&D); for antigen recognition and presentation HLA class II (Dako), CD163, mannose receptor, CD11b (BD biosciences); as pro-inflammatory markers IL-1beta (gift from Dr. Boraschi), TNF-alpha (U-Cytech), IL-6 (Genzyme), IL-12p40/p70 (Pharmingen); for CNS proteins MOG, MAP-2 (Pierce).

### In Vitro Model for Myelin-Driven Foam Cell Formation

Myelin was isolated as described previously (Norton and Poduslo, 1973). In short, white matter derived from post-mortem brain tissue was homogenized in 0.32 M sucrose and subsequently layered on 0.85 M sucrose. After centrifugation at 75,000 g, myelin was collected from the interface, washed in water and suspended in water for osmotic shock. Using this method, the purified myelin was shown to be free of any recognizable fragments of other subcellular elements. Previous studies have shown that purified myelin structurally resembled the whole multilamellar myelin structure surrounding as seen in tissue sections using electron microscopy (Autilio et al., 1964).

Peripheral blood mononuclear cells were isolated from heparinized blood from healthy donors using a Ficoll density gradient. Subsequently, monocytes were purified using Percoll density gradient resulting in >80% monocytes. Monocytes were cultured in suspension at a concentration of 1×10⁶ cells/ml in Teflon flasks (Nalgene) in RPMI with 5% human AB serum. After five to seven days, monocyte-derived macrophages were recovered from the Teflon flasks and seeded in tissue culture plates. After 24 hours, non-adherent cells were removed and remaining cells were >95% macrophages as determined by macrophage-specific esterase staining. Foamy macrophages were generated in vitro by incubating macrophages with myelin for 24 hours to seven days (referred to as one-day and seven-day-old foamy macrophages). In most experiments 50 microg/ml myelin was used. Control macrophages were obtained from the same donor, and not fed with myelin.

### ELISA

To determine cytokine production in culture supernatants of foamy macrophages commercial capture ELISA was performed. TNF-alpha, IL-10 and IL-12p40 were measured in the collected culture supernatants. ELISA was performed according to the manufacturers' guidelines (Biosource). Briefly, polystyrene microtiter wells (Immuno Maxisorp) were coated overnight at 4° C. with monoclonal anti-cytokine capture antibodies. Wells were blocked for two hours at room temperature with PBS/0.5% BSA, followed by washing (0.9% NaCl/0.1% Tween20). Freshly thawed supernatants of the cell cultures and recombinant human cytokine-standards were incubated in duplicates for two hours at room temperature in the presence of a biotinylated second anti-cytokine detection antibody. After washing, wells were incubated with BRP-labeled poly-streptavidin (CLB) for 30 minutes at room temperature. HRP revelation was performed with 3,3′,5,5′-tetramethylbenzidine (TMB) peroxidase (KPL). Color development was stopped by adding equal volume of 1M H₂SO₄. Optical density was measured at 450 nm.

CCL18 levels were measured by sandwich ELISA assay using a commercially available CytoSet (Biosource), consisting of a capture-antibody, a biotinylated detection-antibody, recombinant CCL18 standard and streptavidin-HRP conjugate. Assay conditions were exactly as described by the manufacturer.

### Real-Time Quantitative PCR

To quantify mRNA expression by foamy macrophages total RNA was extracted from cell cultures using the GenElute Mammalian Total RNA kit (Sigma). RNA samples were treated with DNAse I (Invitrogen) to remove any contaminating DNA. Using 1 microg of the total RNA as template, copy DNA (cDNA) was prepared using the AMV Reverse Transcription System (Promega). To determine target gene mRNA expression, real-time quantitative reverse-transcription-PCR was performed using TaqMan technology (PE-Applied Biosystems) as described previously (van der Fits et al., 2003). Target gene expression levels were corrected for GAPDH mRNA levels. Sequences of the PCR primers (PE Biosystems), and fluorogenic probes (Eurogentec) are: forward primer 5′CCTTCCTCCTGTGCC TGATG (SEQ ID NO:1), reverse primer 5′ACAATCTCATTTGAATCAGGAA (SEQ ID NO:2), probe 5′TGCCCGACTCCCTTGGGTGTCA (SEQ ID NO:3) for COX-2; forward primer 5′ACGGCGCTGTCATCGATT (SEQ ID NO:4), reverse primer 5′GGCATTCTTCACCTGCTCCA (SEQ ID NO:5), probe 5′CTTCCCTGTGAAAACAAGAGCAAGGCC (SEQ ID NO:6) for IL-10; forward primer 5′GCCCAGGCAGTCAGATCATC (SEQ ID NO:7), reverse primer 5′-GGGTTTGCTACAACATG GGCT (SEQ ID NO:8), probe 5′CTCGAACCCCGAGTGACAAGCCTG (SEQ ID NO:9) for TNF-α; forward primer 5′CACCGGAACGACATGGAGA (SEQ ID NO:10), reverse primer 5′TCCAGGCGACAAAAGG GTTA (SEQ ID NO:11), probe 5′TGGGCTTCGTCTACTCCTTTCTGGGTC (SEQ ID NO:12) for PGES; forward primer 5′GCCTGGCCTCCAGAAAGACC (SEQ ID NO:13), reverse primer 5′ACCTGGTACATCT TCAAGTCTTCATAAAT (SEQ ID NO:14), probe 5′CTTTTATGATGGCCCTGTGCCTTAGT (SEQ ID NO:15) for IL-12p35; forward primer 5′GCCAGGAGTTGTGAGTTTCCA (SEQ ID NO:16), reverse primer 5′-TGCAAGGCCCTTCATGATG (SEQ ID NO:17), probe 5′TCTGACCACTTCTCTGCCTGCCCA (SEQ ID NO:18) for CCL18, forward primer 5′-GTTCCCCATATCCAGTGTGG (SEQ ID NO:19), reverse primer 5′-TCCTTTGCAAGCAGAACTGA (SEQ ID NO:20), probe TGGCTGTG (SEQ ID NO:21) (Roche) for IL-23p19.

### Statistical Analysis

Statistical analysis was performed using the non-parametric Mann-Whitney analysis. P values<0.05 were considered significant.

Multiple sclerosis (MS) is a chronic inflammatory autoimmune disease of the central nervous system (CNS) and is characterized by the presence of demyelinated areas throughout the CNS (Sospedra and Martin, 2005). Various mechanisms leading to demyelination and axonal suffering have been implicated and the production of toxic inflammatory mediators by infiltrating and resident CNS macrophages is believed to play a pivotal role (Becher et al., 2000; Cannella and Raine, 2004; Lassmann, 2004; Matute and Perez-Cerda, 2005; Raine, 1994; Sospedra and Martin, 2005; Wingerchuk et al., 2001).

Different subsets of myeloid cells have distinct roles in the development of experimental autoimmune encephalomyelitis (EAE), an animal model for MS. These distinct and specialized roles of myeloid cells depend on their origin and, importantly, their location (Greter et al., 2005; Heppner et al., 2005; McMahon et al., 2005; Platten and Steinman, 2005). As such, perivascular cells appear to be optimally positioned for the modulation of infiltrating T-cell activity whereas parenchymal myeloid cells may have a more prominent role in mechanisms involved in myelin breakdown and axonal suffering (Platten and Steinman, 2005).

The plasticity and functional polarization of macrophages have received renewed attention in light of novel key properties of different forms of macrophages. Two extremes of a continuum have been identified for macrophages, being M1, or classically activated macrophages, and M2, or alternatively activated macrophages (Gordon, 2003; Mantovani et al., 2004; Mantovani et al., 2002; Mosser, 2003). The M1 phenotype is typically induced in vitro by IFN-gamma, TNF-alpha or LPS, whereas the M2 phenotype can be induced by IL-10, IL-4 or by the lipid mediator PGE₂, which is a strong inhibitor of pro-inflammatory immune responses (Gratchev et al., 2001; Harris et al., 2002; Hinz et al., 2000; Ikegami et al., 2001; Kalinski et al., 1997). M1 macrophages are characterized by a high production of pro-inflammatory mediators and are involved in Th1 cell responses and killing of micro-organisms and tumor cells. In contrast, M2 macrophages are associated with Th2 responses, scavenging of debris, promotion of tissue remodeling and repair and expression of anti-inflammatory molecules, including IL-1ra (IL-1 receptor antagonist) and CCL18 (Gordon, 2003; Mantovani et al., 2004). CCL18 in particular is a specific marker for human alternatively activated macrophages (Goerdt et al., 1999; Gordon, 2003; Kodelja et al., 1998; Mantovani et al., 2002) and is likely involved in immune suppression. Demyelinating MS lesions are characterized by the presence of foamy macrophages, a characteristic subset of myeloid cells, which acquire their distinctive morphology by ingestion and accumulation of vast amounts of myelin-derived lipids. Foamy macrophages originate from both resident microglia and infiltrating monocytes. Thirty to 80% of foamy macrophages in demyelinating lesions are estimated to be blood-derived (Li et al., 1996). Besides their apparent role in scavenging myelin, it is still poorly understood if and how foamy macrophages may affect the local inflammatory process. Since MS lesions are self-limiting and do not expand indefinitely it is likely that local mechanisms restrict CNS inflammation and may also promote tissue repair. We hypothesized that foamy macrophages are anti-inflammatory M2-type macrophages and actively contribute to the resolution of brain inflammation and hence to tissue integrity and function. Our findings reveal an important and previously overlooked anti-inflammatory and modulatory role for foamy macrophages in MS lesions.

### Results of Example 1

Multiple sclerosis (MS) lesion activity concurs with the extent of inflammation, demyelination and axonal suffering. Proinflammatory myeloid cells contribute to lesion development, but the self-limiting nature of lesions implies as yet unidentified anti-inflammatory mechanisms. We addressed the hypothesis that myelin ingestion by myeloid cells induces a foamy appearance and confers anti-inflammatory function. We show that myelin-containing foam cells in MS lesions consistently express a series of anti-inflammatory molecules while lacking pro-inflammatory cytokines. Unique location-dependent cytokine and membrane receptor expression profiles imply functional specialization allowing for differential responses to micro-environmental cues. A novel human in vitro model of foamy macrophages functionally confirmed that myelin ingestion induces an anti-inflammatory program. Foamy macrophages are unable to respond to prototypical inflammatory stimuli. Preliminary microarray data suggest altered expression of multiple chemokines by foamy macrophages. Ongoing transwell migration experiments indeed show differential migration patterns of and towards foamy macrophages. These findings provide novel insights into the mechanisms of lesion control and may open new roads to intervention.

### Foamy Macrophages Express Anti-Inflammatory Markers and Demonstrate a Unique Location-Dependent Phenotype

To determine the immune phenotype of lipid-laden foamy macrophages in MS lesions, we used antibodies against CNS proteins, various surface markers involved in antigen recognition and presentation, and pro- and anti-inflammatory markers characteristic for M1 and M2 macrophages (Goerdt et al., 1999; Gordon, 2003; Kodelja et al., 1998; Mantovani et al., 2002). Foamy macrophages were defined by their characteristic morphology, strong HLA-DR expression and presence of neutral lipids, which are detected by oil red 0 histochemistry (ORO). To determine whether foamy macrophages display phenotypic and functional specialization dependent on micro-location, we analyzed the phenotype of these cells in different micro-locations. We distinguished between foamy macrophages within the lesion, in perivascular spaces within the lesion and in the outer or inner rim. The distinction between the outer and inner rim was based on the presence of neutral lipids, MOG and on the size of the foamy macrophages. Outer rim foamy macrophages were smaller in size and contained more MOG, but less neutral lipids than inner rim foamy macrophages.

IL-6, a cytokine with pro- as well as anti-inflammatory properties as well as the anti-inflammatory M2 marker IL-1ra and prostaglandin E₂ synthase (PGES) were differentially expressed in the distinct areas of an MS-lesion. Whereas IL-6 and IL-1ra were detected mostly in perivascular and lesional foamy macrophages, PGES was mostly expressed in the outer, and to a lesser extent in the inner rim. Importantly, expression patterns between cells varied even when cells were in close proximity. Mannose receptor, which is characteristic for M2 macrophages (Gordon, 2003; Mantovani et al., 2004; Mantovani et al., 2002; Mosser, 2003), was highly expressed on foamy macrophages in perivascular spaces but was mostly absent on parenchymal foamy macrophages. Occasionally, a weakly positive cell was observed which was always in the vicinity of a blood vessel. TGF-beta expression showed the reverse expression pattern with more pronounced expression by parenchymal foamy macrophages compared to perivascular foamy macrophages.

As hypothesized, the relative levels of expression were related to specific micro-locations within the lesion. Foamy macrophages in the lesion rim contained MOG, and immunoreactivity showed a decreasing trend towards the center of the lesion, possibly reflecting time-dependent myelin degradation. In contrast, intracellular neuronal antigen MAP-2 immunoreactivity increased towards the center of the lesion, implicating that neuronal damage occurs mostly in the lesion center. Only foamy macrophages within perivascular spaces expressed the surface markers CD11b, CD163 and mannose receptor. The anti-inflammatory molecules IL-1ra, CCL18, IL-10, TGF-beta and IL-4 were all strongly expressed by foamy macrophages, and expression was highest in the center of the lesion. Interestingly. IL-10 expression was absent on foamy macrophages in perivascular spaces. The pro-inflammatory cytokines TNF-alpha, IL-1 beta, IL-12p40/70 were not expressed by foamy macrophages in any of the micro-locations, whereas cells associated with vessels in normal appearing white matter (NAWM) did express these pro-inflammatory cytokines. Phenotypic heterogeneity was not observed among non-foamy macrophages which were present in low numbers in perivascular spaces in NAWM.

Thus, we demonstrate that foamy macrophages in the brain have clear anti-inflammatory characteristics, resemble M2 macrophages, and have a unique phenotype depending on the micro-location.

Myelin Induces a Foamy Morphology in Macrophages Resembling that of Foamy Macrophages In Situ

Next, we set out to determine whether ingestion of myelin in vitro results in an anti-inflammatory function of foamy macrophages as observed in situ. Therefore, we first developed a fully human in vitro model of foamy macrophages. In short, human monocyte-derived macrophages are cultured in the absence or presence of human brain-derived myelin for 24 hours. Whereas cells cultured in the absence of myelin did not appear foamy (at magnification 32×), those cultured with myelin acquire a characteristic foamy morphology as observed by light microscopy. Human primary macrophages obtained from healthy donors were fed with 50 microg/ml human myelin and changes in the morphology were monitored by light microscopy and by ORO staining to detect intracellular neutral lipids. Although small individual changes in kinetics between individual donors were observed, macrophages acquired a foamy morphology between 24 and 48 hours and contained a markedly increased number and size of lipid droplets in comparison to control macrophages (i.e., not fed with myelin) as demonstrated by ORO staining. The typical foamy morphology of macrophages could still be observed one week upon the initial addition of myelin. Macrophage viability was not affected by myelin ingestion when a dose range of 1 to 100 microg/ml as was used, as was demonstrated by trypan blue staining.

### Foamy Macrophages do not Mount Pro-Inflammatory Responses to Prototypical Inflammatory Stimuli and Produce Anti-Inflammatory Mediators

To assess the effect of myelin ingestion on macrophage function, cytokine levels were determined in supernatants of myelin-laden macrophages before and after LPS stimulation. Since variation in myelin lipid composition between MS and normal brain has been reported (Woelk and Borri, 1973), myelin was isolated from white matter of three control brains and three MS brains to investigate possible functional differences. Macrophages were incubated with the distinct myelin preparations for 24 hours and IL-10 and IL-12p40 levels were determined in the supernatants by ELISA. None of the myelin preparations induced IL-12p40 and only the highest dose of one MS brain-derived myelin was associated with a transient IL-10 induction. All myelin preparations inhibited LPS-induced IL-12p40 and IL-10 induction in a dose-dependent fashion. No significant differences were observed in cytokine production between foamy macrophages generated using the different myelin preparations. For subsequent experiments 50 microg/ml myelin was used.

Next, the effect of myelin ingestion on LPS-induced mRNA levels of different pro- and anti-inflammatory mediators was determined. Macrophages were incubated with myelin for 24 hours and subsequently stimulated with LPS for an additional two hours, after which RNA was isolated and real time RT-PCR was performed for IL-12p35, TNF-alpha, IL-10, COX-2, PGES and CCL18. LPS-induced IL-12p35 and TNF-alpha expression by foamy macrophages was completely inhibited. IL-10 was slightly but not significantly induced by LPS in control macrophages as well as foamy macrophages. COX-2 was increased after LPS stimulation in control macrophages but this induction was not significantly inhibited in foamy macrophages. Foamy macrophages showed between 15 to 50 and eight- to twelve-fold induction of CCL18 and PGES compared to control macrophages. Thus, myelin ingestion resulted in a differential modulation of LPS responses. LPS-induced IL-12p40 and TNF-alpha expression was strongly and significantly inhibited, IL-10 and COX-2 expression remained unaffected and the expression of anti-inflammatory CCL18 and PGES significantly increased.

To determine whether myelin ingestion results in long-term modulation of macrophage function, macrophages were incubated with myelin for the indicated time periods and real time RT-PCR was performed for IL-12p35, IL-10, PGES and CCL18. IL-10 mRNA was not detectable at any time point. After myelin uptake, IL-12p35 expression was decreased, albeit not significantly, over time in comparison to control macrophages. In contrast to IL-12p35 both PGES and CCL18 were induced by myelin. Seven-day-old foamy macrophages expressed ten- and ninety-fold more PGES and CCL18 than control macrophages. IL-12p40, IL-10, and CCL18 levels were subsequently determined in supernatants of these foamy macrophages. CCL18 is constitutively produced by macrophages and production by foamy macrophages is increased at day seven after myelin ingestion, paralleling the increased CCL18 mRNA expression by foamy macrophages. IL-12p40 and IL-10 were not detectable.

Subsequently we determined whether the aberrant LPS response persisted over time. Seven days after initial myelin ingestion, foamy macrophages were stimulated with 1 ng/ml LPS for 24 hours and cytokine levels in the supernatant were determined by ELISA. LPS-induced IL-12p40 and IL-10 production by these foamy macrophages was abolished completely whereas CCL18 was significantly increased. In addition, responses to other prototypical pro-inflammatory stimuli, such as peptidoglycan and zymosan, were also completely abolished.

The relapsing-remitting nature of MS strongly suggests the presence of potent counter-regulatory mechanisms that keep the disease in check. One such mechanism may be the active control of inflammation in the CNS itself thus preventing infinite expansion of the demyelinating lesion. Inflammation and demyelination are responsible for at least short-term neurological symptoms. Inflammation probably contributes to axonal loss as neurons are more vulnerable to environmental insults when the protective myelin sheaths are destroyed and the axons exposed (Grigoriadis et al., 2004; Kuhlmann et al., 2002). It is, therefore, imperative that in the developing lesions the production of toxic molecules is halted and that inflammation is limited allowing for tissue repair (Sospedra and Martin, 2005). Myelin-laden foamy macrophages are abundantly present in demyelinating lesions and although it is generally assumed that these cells contribute to inflammation, evidence for this is scarce (van der Laan et al., 1996). This lack of data on foamy macrophage function in MS is in sharp contrast with the increasing attention for foam cells in atherosclerosis (Greaves and Gordon, 2005) reporting potent immune-regulatory functions by lipids and lipid-induced molecules (Harris et al., 2002; Joseph et al., 2004; Joseph et al., 2003; Lawrence et al., 2002; Pettus et al., 2002). Lipid-laden cells are anti-inflammatory (Lawrence et al., 2002) and it was shown that low-density lipoprotein (LDL) uptake by macrophages inhibits TNF-induced TNF expression and induces IL-10 (Ares et al., 2002; Lo et al., 1999; Varadhachary et al., 2001). Foamy macrophages in the rim of active demyelinating lesions have been shown to contain plasma LDL (Newcombe et al., 1994).

Here, we establish that foamy macrophages in active MS lesions have consistent immunosuppressive function, while displaying a unique surface phenotype dependent on the micro-location. In addition, we demonstrate that ingestion of human myelin alters human macrophage function in vitro by inducing anti-inflammatory molecules and by inhibiting responses to pro-inflammatory stimuli. The results presented here reveal a new regulatory pathway in MS.

We demonstrate that foamy macrophages in demyelinating lesions in MS brain express various markers that are involved in anti-inflammatory processes, including IL-1ra, IL-10, CCL18, TGF-alpha, and that a subset of the foamy macrophages express markers involved in innate immunity, including mannose receptor and CD163. These molecules are all characteristic for alternatively activated M2 macrophages (Gordon, 2003; Mantovani et al., 2002; Mosser, 2003) and this strongly suggests a local regulatory immunosuppressive role. Importantly, our data show that foamy macrophages occur in discrete subsets. This may reflect their origin (i.e., microglial-derived vs. blood-derived), their age and the degree of lipid degradation, and most likely the cues received from their microenvironment. These cues include the type of ingested lipids, cytokine environment, presence and identity of neighboring cells or signals from the extracellular matrix. The unique combination of surface and intracellular molecules of individual macrophages in different areas of the lesion suggests that they are likely to exert diverse functions depending on their location. Foamy MOG-positive macrophages in the lesion rim may be more involved in phagocytosis of myelin whereas foamy macrophages inside the lesion appear to be geared for down-regulation of inflammation as suggested by high expression of anti-inflammatory cytokines. Interestingly, our in vitro data show a transient increase in PGES expression and a sustained increase in CCL18 expression. This parallels the in situ analysis showing highest expression of PGES in foamy macrophages in the lesion rim that likely have ingested myelin more recently than foamy CCL18-positive macrophages in the lesion center. IL-10 was expressed in situ mostly by lesional foamy macrophages. In vitro IL-10 is transiently induced by myelin, but LPS-induced IL-10 production is inhibited. This suggests complex regulation of IL-10 expression both in vitro and in vivo which will need to be explored in more detail in future studies. Regulatory foamy macrophages in perivascular spaces are likely to affect the function of newly infiltrating cells. Current experiments employ genomic as well as well as biochemical approaches to identify such immunomodulating mechanisms.

We show here that the observed functional phenotype of foamy macrophages in MS lesions results from the accumulation of lipids derived from myelin and phagocytozed apoptotic cell membranes, in concert with local microenvironmental cues, such as differences in extracellular matrix content in the perivascular infiltrate versus the lesion in the brain parenchyma. Foamy macrophages demonstrate a phenotype resembling that of anti-inflammatory M2 macrophages, are likely to contribute to resolution of inflammation, and may therefore be responsible for inhibiting further lesion development and promoting lesion repair. In addition, they may also function as a first line of defense against infiltrating inflammatory myeloid cells. Future studies are required to elucidate which lipid components are able to regulate macrophage function and which mechanisms are involved. Understanding the mechanisms behind naturally occurring counter-regulatory processes allows for definition of new cellular targets for therapeutic drug design for the treatment of MS and even has broader applications for other foam cell-associated diseases including atherosclerosis and lung conditions.

### Example 2

### Do Compounds Modulate Immune Responses by Macrophages and Foam Cells?

### Experimental Design:

Human monocyte-derived macrophages were cultured in medium (=macrophages) or in the presence of human brain-derived myelin for 48 hours (=foam cells).

Macrophages and foam cells were cultured in the presence of 10 microg/ml compounds BTMP1, BTMP2, BTMP3, BTMP4, BTMP5, BTMP6, BTMP7, BTMP8, BTMP9, BTMP10 for three hours. Compounds were tested under cover by order of Biotempt BV, Hoge Linthorst 1, 7958 NZ, The Netherlands.

10 ng/ml LPS was added to the cultures for an additional 16 hours.

Supernatants were collected and ELISA performed for TNF-alpha, IL-12p40, and IL-10.

### Results:

Protein levels are depicted in Table 2.

LPS-induced TNF-alpha, IL-12p40 and IL-10 in macrophages as expected, confirming the experimental system performed as usual.

Foam cells demonstrated decreased LPS responses for IL-10 and IL-12p40 as expected. LPS-induced TNF-alpha production by foam cells was not affected as has been observed before.

Effects of compounds on LPS responses are shown in Table 1.

The compounds did not affect macrophage or foam cell morphology or viability as judged by microscopic examination.

### Example 3

### Do Compounds Affect Cytokine Production by Human Macrophages and Foam Cells?

### Experimental Design:

Human monocyte-derived macrophages from a healthy blood bank donor were cultured in medium (=macrophages) or in the presence of human brain-derived myelin for 48 hours (=foam cells).

Macrophages and foam cells were cultured in duplicate in the presence of 10 microg/ml of compounds BTMP1, BTMP2, BTMP3, BTMP2, BTMP5, BTMP6, BTMP7, BTMP8, BTMP9, BTMP10 for two or eight hours, or cultured in macrophage medium with vehicle.

Cells were lysed and real time RT-PCR (TaqMan technology) was performed on all samples for GAPDH (housekeeping gene), TNF-alpha (pro-inflammatory), IL-12p35 (pro-inflammatory), IL-10 (anti-inflammatory), CCL18 (chemokine), COX-2 (prostaglandin pathway).

### Results:

Effects of compounds on mRNA expression levels are depicted in Tables 2, 3 and 4.

The compounds did not affect macrophage or foam cell morphology or viability as judged by microscopic examination.

cDNA quality of two samples was not sufficient for reliable semi-quantification. Values of these samples (#6, compounds two hours on macrophages; #10, compounds eight hours on foam cells) have been omitted.

### Example 4

### Do Foam Cells Differentially Express Chemokines Compared to Control Macrophages?

### Experimental Design:

Human monocyte-derived macrophages from a healthy blood bank donor were cultured in medium (=macrophages) or in the presence of human brain-derived myelin for 48 hours (=foam cells).

10 microg/ml of the compounds BTMP1, BTMP2 or BTMP9 was added to macrophages or foam cells for six hours, or macrophages or foam cells were cultured in macrophage medium with vehicle.

Cells were lysed, RNA isolated and Affymetrix microarray (U133+2 chip with 53.675 transcripts) was used according to the manufacturers instructions to determine relative mRNA levels.

### Results:

Effect of myelin ingestion and additional effect of the compounds BTMP1, BTMP2 or BTMP9 on eight different selected chemokines is depicted in Table 6.

Compounds did not affect the chemokine expression by control macrophages.

### Example 5

### Effect of Myelin on Liver X Receptor (LXR)-Mediated Pathways in Foamy Macrophages

Liver X receptors are nuclear receptors that are involved in the control of cholesterol efflux. In addition to their function in lipid metabolism, LXRs have also been found to modulate immune and inflammatory responses in macrophages. Synthetic LXR agonists promote cholesterol efflux and inhibit inflammation in vivo (N. Zelcer and P. Tontonoz, 2006, Liver X receptors as integrators of metabolic and inflammatory signaling, J. Clin. Invest. 116:607). There are two isoforms of LXR known, LXRα and LXRβ, which are both expressed in macrophages. LXRs can be activated by oxysterols, which are metabolites of cholesterol. Activation of LXR results in the induction of several ABC transporters (e.g., ABCA-1), HDL remodeling enzymes (e.g., cholesterol ester transfer protein and phospholipid transfer protein) and apoE (extracellular acceptor for cholesterol). Activation of LXR by oxysterols also inhibits the expression of pro-inflammatory molecules, such as inducible nitric oxide synthase (iNOS), cyclooxygenase 2 (COX-2), IL-6, IL-1β, G-CSF, MCP-1 (CCL-2), macrophage inflammatory protein 1β (MIP-1β/CCL-4) and matrix metalloproteinase 9 (MMP-9) through the inhibition of NFκB (S. B. Joseph, A. Castrillo, B. A. Laffitte, D. J. Mangelsdorf and P. Tontonoz, 2003, Reciprocal regulation of inflammation and lipid metabolism by liver X receptors, Nat. Med. 9:213). LXRs can also interfere with the expression of other transcription factors, (i.e., c-fos and phospho-c-jun), and subsequently inhibit genes that contain an AP-1 promoter, such as TNF-α and osteopontin, a cytokine and monocyte adhesion molecule (M. P. Ares, M. Stollenwerk, A. Olsson, B. Kallin, S. Jovinge, and J. Nilsson, 2002, Decreased inducibility of TNF expression in lipid-loaded macrophages, BMC Immunol. 3:13; D. Ogawa, J. F. Stone, Y. Takata, F. Blaschke, V. H. Chu, D. A. Towler, R. E. Law, W. A. Hsuch and D. Bruemmer, 2005, Liver X receptor agonists inhibit cytokine-induced osteopontin expression in macrophages through interference with activator protein-1 signaling pathways, Circ. Res. 96:e59).

### Hypothesis

LXRs mediate myelin-induced inhibition of inflammatory responses by myeloid cells.

More specifically, Myelin or myelin metabolites bind to and activate LXRs, resulting in increased expression of LXR-induced genes and inhibition of inflammatory pathways, (e.g., the NF-κB pathway).

### Experiments

1. Does Ingestion of Myelin Result in Altered Expression of LXRs and LXR-Induced Genes by Human Macrophages?

Human primary macrophages were incubated for 24 to 48 hours with myelin and subsequently stimulated with 1-10 ng/ml LPS for two or six hours. Relative mRNA expression levels of LXR-alpha and its target gene ABCA1 were determined.

### Results:

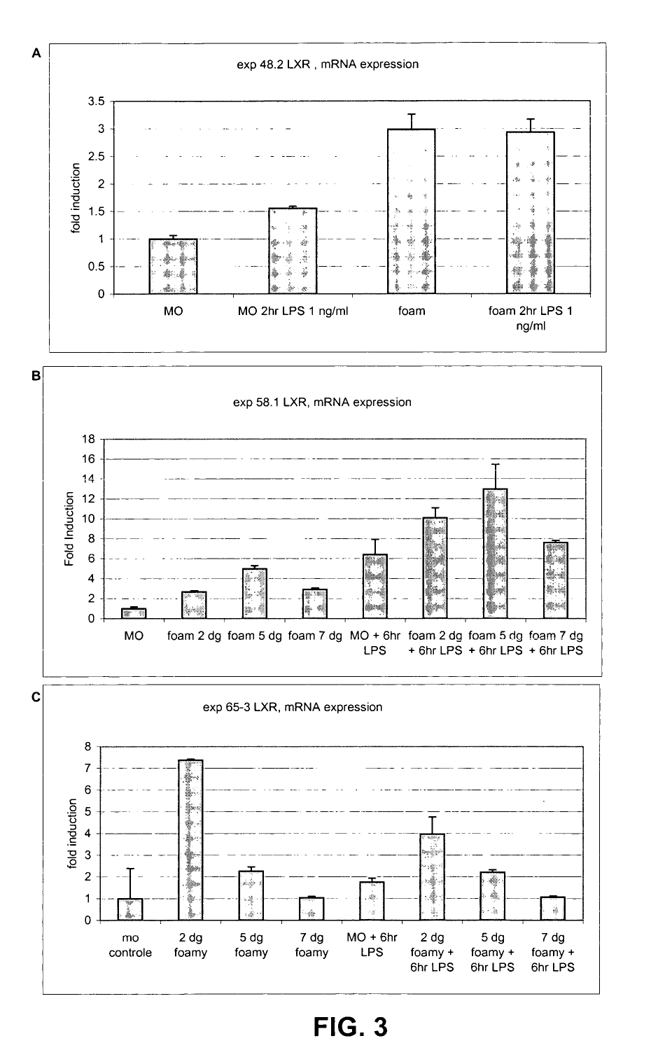

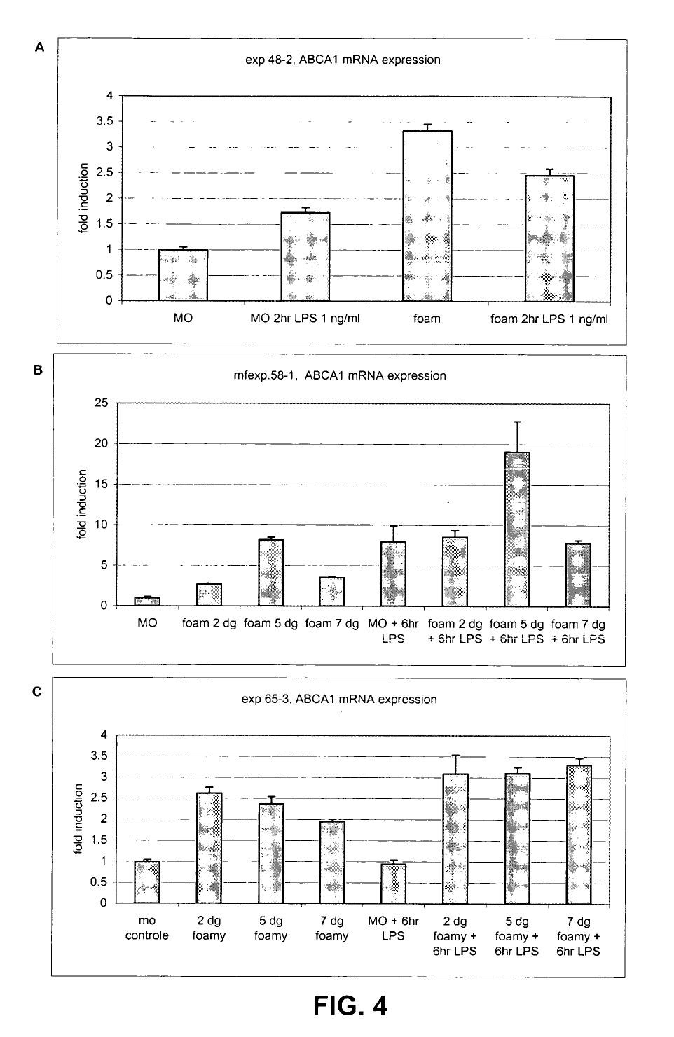

Both genes were significantly induced in foamy macrophages as compared to control macrophages. FIG. 3 shows relative LXR-alpha mRNA expression in three experiments using different donors. FIG. 4 shows relative ABCA1 mRNA expression levels in three experiments using different donors.

### 2. Is the Expression of LXR-Alpha and its Target Gene ABCA1 Altered in Ms Brain as Compared to Brain of Non-Demented Controls?

RNA was isolated from brain tissue containing lesions (e.g., pre-active, active demyelinating, chronic inactive) of seven MS patients, brain tissue consisting of normal appearing white matter of four MS patients (MS patients 5, 9, 10, 11), and white matter of six control individuals. Semi-quantitative RT-PCR was performed for LXRalpha and ABCA1 using GAPDH as a house-keeping gene.

### Results:

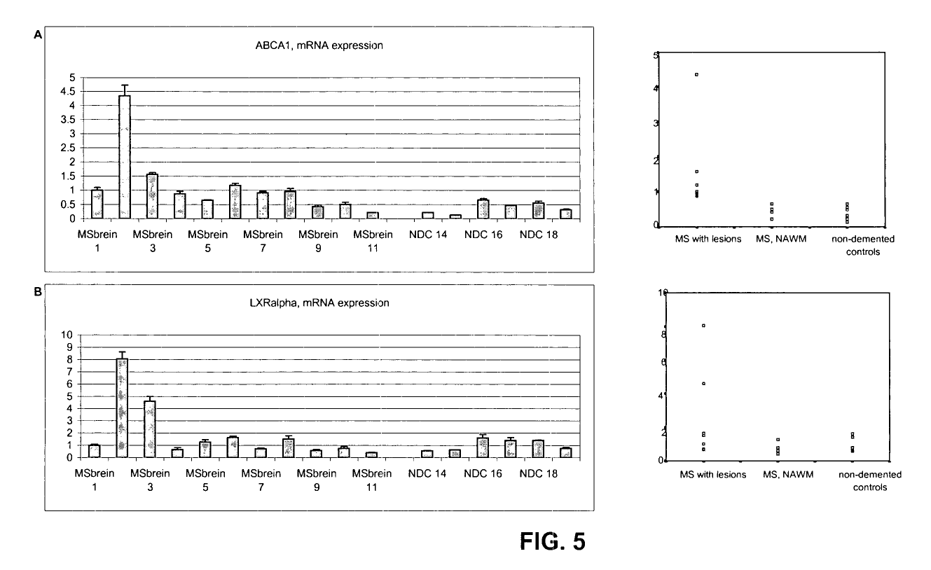

LXR-alpha mRNA was increased, but not significantly, in MS brain tissue with lesions, as compared to NAWM MS brain, or non-demented controls (FIG. 5)

ABCA1 was significantly increased in MS brain tissue with lesions, as compared to NAWM MS brain, and non-demented controls (FIG. 5, Kruskal Wallis p=0.003).

The brain tissue of the two MS patients with the highest LXR-alpha and ABCA1 mRNA expression contained the highest number of foamy macrophages as determined by immunohistochemical analysis.

LXR-alpha expression correlated strongly with ABCA1 expression among the individuals (FIG. 6, Spearman: p<0.01).

### Planned Experiments:

### 1. Hypothesis: Foamy Macrophages are the Source of ABCA1 and ApoE in MS Brain Tissue.

Experiment: Immunohistochemical analysis ABCA1 expression in brain tissue of MS patients to determine the source of ABCA1 and ApoE.

### 2. Hypothesis: LXRs Mediate the Myelin-Mediated Abrogation of LPS Responses as Described in Boven et al., Brain 2006

Experiment: Generate foamy macrophages using from bone marrow of LXR-null mice and WT mice. Stimulate foamy macrophages with LPS and determine levels of LXR target genes (e.g., ABCA1), and expression and production of pro- and anti-inflammatory mediators (e.g., IL-12, TNF-alpha, IL-10)

### Example 6

### Reciprocal Regulation of Adenosine Receptor Expression on Foamy Macrophages In Vitro and in MS Lesions

Adenosine is released at sites of inflammation and tissue damage and activates adenosine receptors. Whereas many of the reported adenosine receptor-mediated effects are neuroprotective, adenosine may also aggravate neuronal injury by promoting inflammation.

We have recently described an anti-inflammatory function for foamy macrophages in multiple sclerosis (MS) lesions and hypothesized that expression of adenosine receptors is altered in these cells thereby affecting their function. Therefore, we determined mRNA expression levels of the adenosine receptors (A1, A2a, A2b, and A3) in human foamy macrophages and in MS lesions. A3 receptor mRNA expression was significantly down-regulated (five-fold) in foamy macrophages as compared to control macrophages. After LPS stimulation, A2a mRNA expression in foamy macrophages was strongly up-regulated (100-fold). In contrast, A3 receptor mRNA was further down-regulated (100-fold). To assess whether this potent mRNA regulation also occurs during MS, expression levels were determined in MS brain and brain tissue of non-demented control. Surprisingly, A3 receptor mRNA was significantly up-regulated in MS brain and the expression correlated with lesion activity. Immunohistochemistry will reveal which cells are expressing A3 receptors in MS brain. Expression of other adenosine receptors was not altered.

Our data demonstrate that adenosine receptor expression on foamy macrophages in the brain is tightly and potently regulated. The changed balance of the expression levels of the different adenosine receptors is likely to influence the functional response to adenosine. Understanding the regulation of these immune-modulatory receptors will allow better understanding of endogenous neuroprotective mechanisms and will thereby open new roads to disease intervention.

### Background

Multiple sclerosis is a chronic disease of the central nervous system, characterized by inflammation, demyelination and neuronal damage. Local inflammation and tissue damage in the CNS is likely to result in the release of adenosine, which can subsequently activate adenosine receptors. Whereas many of the reported adenosine receptor-mediated effects are neuroprotective, adenosine may also aggravate neuronal injury by promoting inflammation. The various effects of adenosine are mediated by four different adenosine receptors (A1, A2a, A2b, and A3 receptors). The local balance in expression levels of the different adenosine receptors may very well dictate the response to adenosine. Although various studies describe the effect of adenosine on individual receptors, not much is know about the response to adenosine by cells with a specific combination of the different adenosine receptors.

We have recently described an anti-inflammatory function for foamy macrophages in multiple sclerosis (MS) lesions. We now hypothesized that the expression profile of the four adenosine receptors is altered in these cells, resulting in a potent anti-inflammatory response to adenosine by foamy macrophages.

### Experiments

### 1. Does Ingestion of Myelin Result in Altered Expression of Adenosine Receptors by Human Macrophages?

Human primary macrophages were incubated in the presence of human brain-derived myelin to generate foamy macrophages. After 48 hours, myelin was washed away and cells were cultured for the indicated time points. Macrophages and foamy macrophages were subsequently cultured in the absence or presence of LPS for six hours. Then, RNA was collected and semi-quantitative RT-PCR was performed for A1, A2a, A2b, and A3 receptors using GAPDH as a house-keeping gene.

### Results:

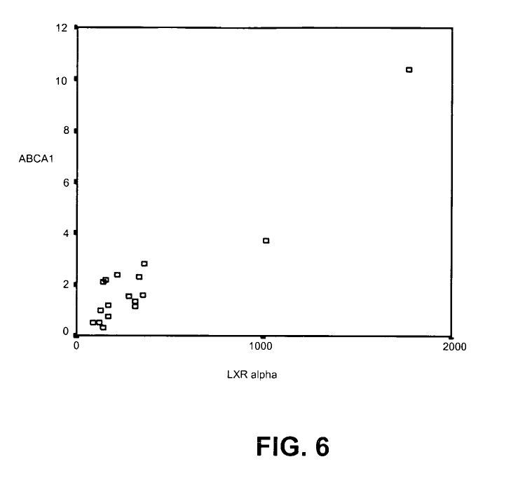

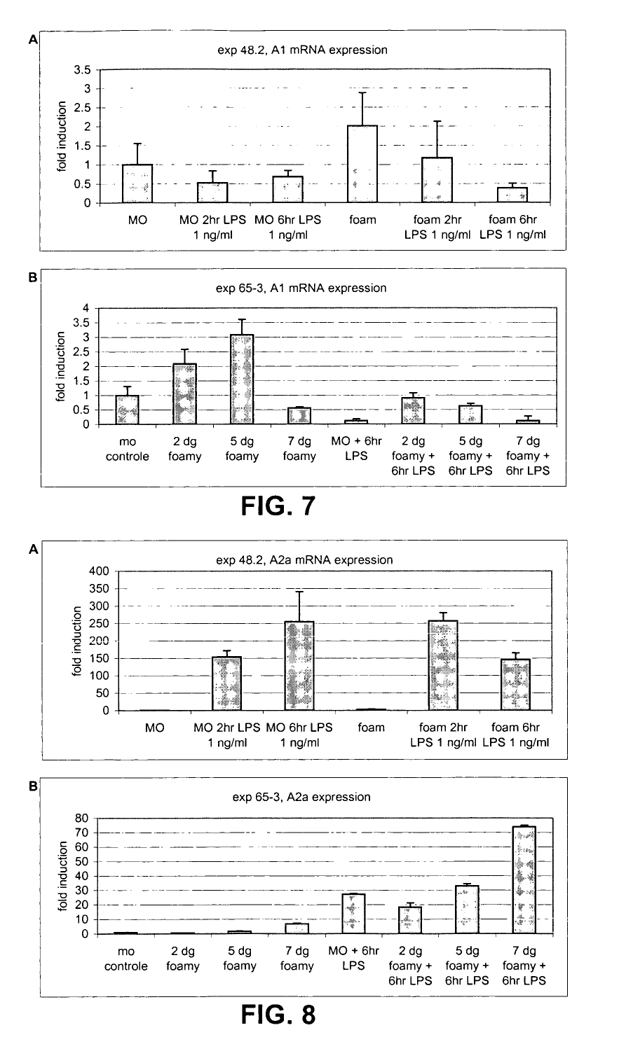

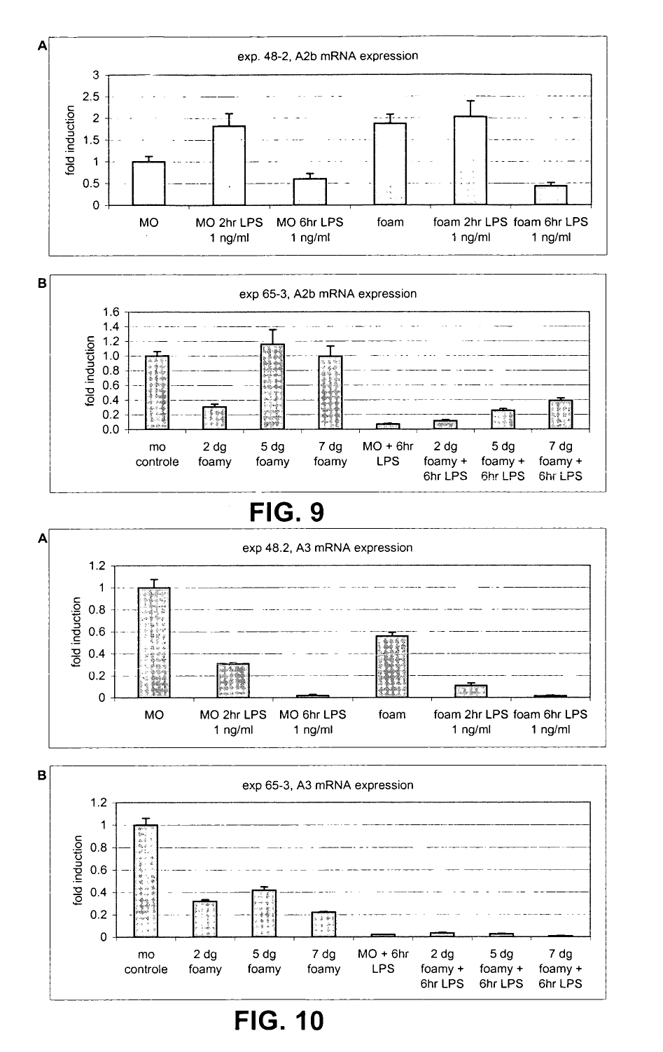

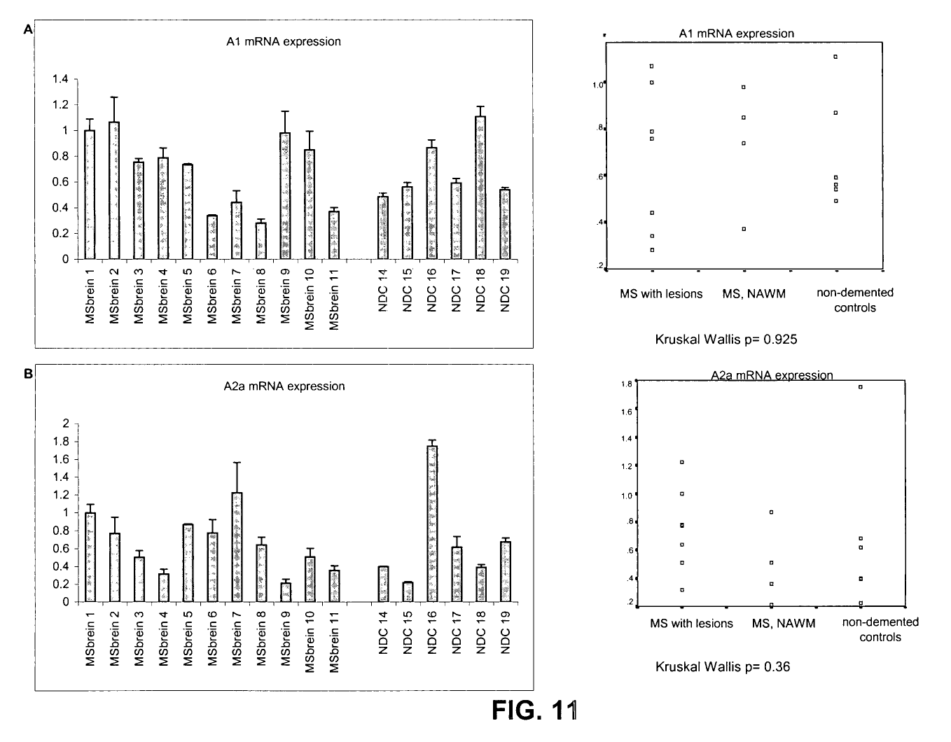

FIGS. 7-10 show mRNA expression levels of the A1, A2a, A2b, and A3 receptors, respectively. Foamy macrophages demonstrate significantly decreased levels of A3 mRNA (five-fold reduction) as compared to control macrophages. After LPS stimulation, A2a levels increased in both control as foamy macrophages, whereas A3 mRNA levels decreased even further (100-fold). No significant changes were observed for A1 and A2b expression.

### 2. Is the Expression of Adenosine Receptors Altered in Ms Brain as Compared to Brain of Non-Demented Controls?

RNA was isolated from brain tissue containing lesions (e.g., pre-active, active demyelinating, chronic inactive) of seven MS patients, brain tissue consisting of normal appearing white matter (NAWM) of four MS patients, and white matter of six control individuals. Semi-quantitative RT-PCR was performed for A1, A2a, A2b, and A3 receptors using GAPDH as a house-keeping gene.

### Results:

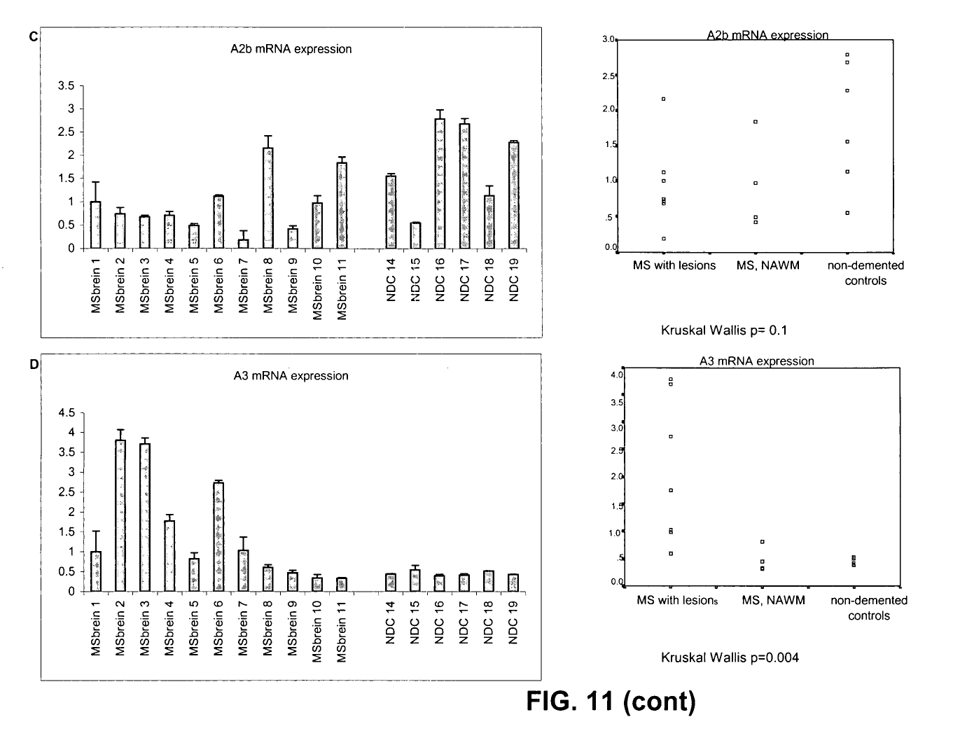

FIG. 11 depicts the relative expression levels in the individual patients. Brain tissue of patients 5, 9, 10, 11 only contained NAWM. FIG. 12 shows scatter plots of the three groups. No significant differences were observed for A 1, A2a, A2b mRNA expression. A3 receptor expression was significantly increased in the group of MS patients with lesional activity (Kruskal Wallis test, p<0.005).

### Further Methods: Quantitation of Lipid Uptake In Vitro

The amount of intracellular lipids at a given time point (so the amount taken up from the environment, plus lipids produced intracellularly, hence reflecting degradation capacity of the given cell) by cells leading to foam cell formation can be reliably quantitated in several ways, including: Counting lipid droplet number in cells on glass slides, stained by Oil red O. Combining this with automated morphometry for counting number of droplets, evaluating their diameter, and expressing this on a per cell basis, and on a cell surface basis taking increasing or decreasing cell size into account.

Fluorometric measurement at 572 nm of lipid stained by Nile Red (other name is AdipoRed) using a fluorometer.

Measurement by flow cytometry using fluorescence of lipids themselves; or by using dyes such as Nile Red, ORO and Sirus red; or by fluorescent labeling of lipid sources such as myelin by DiI or DiO; or by using antibodies against lipid (e.g., oxLDL, sulfatide) or protein (e.g., MBP, MOG, ApoE) components for intracellular staining.

### Source and Nature of Substances Inducing Foam Cell Morphology and Affecting Phagocyte Function

Source and nature of foam-cell-inducing substances may be selected also from complete myelin windings as produced by oligodendrocytes, hence complex particulate multilamellar structures composed of lipids and proteins, perhaps even containing (parts of) axons. These may come available in vivo as large fragments, and/or micelles including small fragments, and/or as soluble compounds. In vitro, the preferred human MS myelin to feed to cells is the complete windings in particulate multilamellar form but lacking the axons. Alternatively, this preparation may be treated with chloroform, thereby dissolving the winding structures and presenting the lipids in liposomes. The myelin may be opsonized by antibody plus complement, and molecules from collectin, pentraxin, ficolin families (e.g., C-reactive protein-CRP, mannose binding lectin-MBL As in vivo in patients, the main culprit is thought to be oxidized LDL (oxLDL). This can be made and used in vitro as well, for example, by CuSO4 oxidation. In addition, acetylated LDL (acLDL) is a well accepted tool in the laboratory mimicking oxLDL. Again, lipids may be opsonized by specific antibody plus complement, and molecules from collectin, pentraxin, ficolin families (e.g., C-reactive protein-CRP, mannose binding lectin-MBL). Different types of lung surfactants may induce foam cell formation in patients and in vitro. Other possible uses for a method as provided herein can be found in the following. Surface phenotyping with sets of existing antibodies against antigen presenting molecules (e.g., MHC-I, MHC-II, CDI); activating and inhibitory costimulatory molecules (e.g., CD40, CD80, CD86, OX-40L and a rational selection from an additional 40 known and testable molecules, dependent on disease under investigation); antigen receptors (e.g., CD14, scavenger receptors, dectin TLRs, CLRs and a further rational selection from an additional 30 known receptors) surface marker sets providing indirect information on M1 versus M2 identity and function (from a set of 20); molecules involved in lipid metabolism including influx and efflux (e.g., CD36, ABCA1, ABCG1); chemokines, chemokine receptors and adhesion molecules involved in migration.

Determining drug functional activity by quantitative assessment of cytokine production on protein and mRNA level, in the absence or presence of prototypical TLR ligands (e.g., LPS, peptidoglycan), CLR ligands (mannose), and scavenger receptor ligands (e.g., oxLDL). In addition by intracellular flow cytometry for these compounds and receptors. Chemokines, chemokine receptors and adhesion molecules involved in migration. Function of molecules involved in export of lipids/metabolites (e.g., ABCA1, ABCG1), and in lipid versus inflammatory responsiveness (e.g., LXRs).

Determining drug functional activity by intracellular enzymes, either by using specific antibody or by using specific substrates to visualize their functional activity: arginase, myeloperoxidase (MPO), inducible nitrix oxide synthase (iNOS), indoleamine 2,3-dioxygenase (IDO), lysozyme, hemoxygenase (HO), N-acetyl muramyl L-alanine amidase (NAMLAA), chitinases (e.g., chitotriosidase).

Determining drug functional activity by in terms of uptake of antigens, lipids, by different cell biological processes. Uptake of dyes (e.g., Lucifer Yellow). Uptake of fluorescent protein antigen, such as BSA-FITC, OVA-FITC, OVA-BODIPY (fluorescence quenched, and activated by cellular uptake and subsequent processing). Uptake of artificial beads, and live or killed microbes (including Candida albicans), quantitated by counting particles or measuring fluorescence. Determining drug functional activity in terms of in vitro intracellular killing activity upon uptake of live microbes. Determining drug functional activity in terms of in vitro migration in several operational systems using fluorometer. Spontaneous migration, and in response to general (e.g., PMA/ionomycin), microbial (fMLP, LPS) and chemokine stimuli (e.g., MCP-1 or CCR7 ligand). Determining drug functional antigen specific activity by in vivo in terms of induction of T- and B-cell reactivity. Operational systems include mice transgenic for the T-cell receptor (TCR) for OVA peptide in context of MHC-I (restricting CD8+ cytotoxic T-cells) or MHC-II (CD4+ T helper cells, and T regulatory cells=Treg).

Other options for use of a method according to the invention are assessment by adoptive transfer of foam cells of healthy donors or patients into mouse models, creating a mouse-human system, by applying:
a. mice irradiated to the extent that all leukocytes have been depleted (“lethally irradiated”) using a dose of XXX Gray.
b. SCID (severe combined immunodeficiency) mice lacking T and B-lymphocytes
c. RAG deficient mice lacking T and B lymphocytes.
d. RAG-common cytokine gamma chain double deficient mice lacking lymphocytes as well as NK cells.

TABLE 2
LPS-induced cytokine responses by compounds tested in
human macrophages and in foam cells

| | mean | mean | sd | sd |
|---|---|---|---|---|
| | macrophages | foam cells | macrophages | foam cells |
| TNF-alpha | | | | |
| (pg/ml) | | | | |
| compound | | | | |
| None | 21664 | 29366 | 2532 | 2733 |
| BTMP1 | 8336 | 16464 | 322 | 2331 |
| BTMP2 | 9075 | 15895 | 1688 | 723 |
| BTMP3 | 13281 | 15895 | 2009 | 5225 |
| BTMP2 | 14475 | 14531 | 482 | 563 |
| BTMP5 | 13054 | 15839 | 2170 | 5626 |
| BTMP6 | 14816 | 19249 | 3697 | 804 |
| BTMP7 | 13167 | 20101 | 402 | 563 |
| BTMP8 | 10723 | 20670 | 4823 | 6189 |
| BTMP9 | 5381 | 13565 | 1125 | 482 |
| BTMP10 | 4812 | 16691 | 643 | 884 |
| IL-10 | | | | |
| (pg/ml) | | | | |
| compound | | | | |
| None | 4140 | 485 | 118 | 29 |
| BTMP1 | 2203 | 222 | 358 | 29 |
| BTMP2 | 2977 | 186 | 105 | 0 |
| BTMP3 | 2793 | 191 | 65 | 2 |
| BTMP2 | 2078 | 179 | 110 | 0 |
| BTMP5 | 2399 | 206 | 18 | 2 |
| BTMP6 | 3105 | 235 | 251 | 11 |
| BTMP7 | 3004 | 229 | 107 | 16 |
| BTMP8 | 2654 | 153 | 11 | 20 |
| BTMP9 | 3654 | 572 | 96 | 13 |
| BTMP10 | 4209 | 601 | 172 | 22 |

| IL-12p40 | | |
|---|---|---|
| (pg/ml) | mean | mean |
| compound | macrophages | foam cells |
| None | 2258 | 1320 |
| BTMP1 | 2038 | 1100 |
| BTMP2 | 1527 | 1093 |
| BTMP3 | 1799 | 899 |
| BTMP2 | 1942 | 997 |
| BTMP5 | 1910 | 912 |
| BTMP6 | 2325 | 804 |
| BTMP7 | 1901 | 1218 |
| BTMP8 | 2426 | 1284 |
| BTMP9 | 3364 | 1274 |
| BTMP10 | 1859 | 1742 |

TABLE 3
Taqman results

| | CCL18 | | | CCL18 | | |
|---|---|---|---|---|---|---|
| | compound treatment | | | compound treatment | | |
| | 2 hours | | | 8 hours | | |
| | | mean | s.d. | | mean | s.d. |
| macrophages | None | 0.99 | 0.45 | none | 1.14 | 0.29 |
| | BTMP1 | 1.00 | 0.13 | BTMP1 | 1.28 | 0.50 |
| | BTMP2 | 1.11 | 0.39 | BTMP2 | 2.42 | 0.16 |
| | BTMP3 | 1.70 | 0.52 | BTMP3 | 1.29 | 0.14 |
| | BTMP4 | 1.00 | 0.43 | BTMP4 | 1.91 | 0.25 |
| | BTMP5 | ND | | BTMP5 | 1.46 | 0.20 |
| | BTMP6 | 1.74 | 0.14 | BTMP6 | 1.13 | 0.39 |
| | BTMP7 | 2.50 | 0.32 | BTMP7 | 0.98 | 0.48 |
| | BTMP8 | 1.31 | 0.20 | BTMP8 | 2.20 | 0.48 |
| | BTMP9 | 2.41 | 1.04 | BTMP9 | 2.29 | 0.53 |
| | BTMP10 | 1.33 | 0.31 | BTMP10 | 1.91 | 0.10 |
| foam cells | None | 278.87 | 99.48 | none | 21.07 | 3.08 |
| | BTMP1 | 886.13 | 353.99 | BTMP1 | 81.82 | 9.06 |
| | BTMP2 | 600.00 | 211.58 | BTMP2 | 52.94 | 11.11 |
| | BTMP3 | 219.51 | 33.08 | BTMP3 | 92.32 | 31.57 |
| | BTMP4 | 355.94 | 81.34 | BTMP4 | 118.82 | 29.83 |
| | BTMP5 | 262.81 | 65.57 | BTMP5 | 124.05 | 24.11 |
| | BTMP6 | 277.78 | 94.19 | BTMP6 | 42.65 | 0.00 |
| | BTMP7 | 205.43 | 46.94 | BTMP7 | 49.25 | 3.90 |
| | BTMP8 | 278.99 | 2.01 | BTMP8 | 55.00 | 16.88 |
| | BTMP9 | 488.87 | 38.70 | BTMP9 | ND | |
| | BTMP10 | 153.76 | 8.86 | BTMP10 | 32.86 | 8.42 |

TABLE 4
Taqman results

| | COX-2 | | | COX-2 | | |
|---|---|---|---|---|---|---|
| | compound treatment | | | compound treatment | | |
| | 2 hours | | | 8 hours | | |
| | | mean | s.d. | | mean | s.d. |
| macro- | None | 0.591211 | 0.226438 | none | 0.94 | 0.32 |
| phages | BTMP1 | 1.28552 | 0.590098 | BTMP1 | 1.47 | 1.33 |
| | BTMP2 | 1.009161 | 0.171062 | BTMP2 | 0.71 | 0.03 |
| | BTMP3 | 1.047836 | 0.344225 | BTMP3 | 1.42 | 0.10 |
| | BTMP4 | 1.28199 | 0.434554 | BTMP4 | 1.58 | |
| | BTMP5 | ND | | BTMP5 | 2.40 | 1.05 |
| | BTMP6 | 1.321293 | 0.966936 | BTMP6 | 0.71 | 0.39 |
| | BTMP7 | 1.01643 | 0.301701 | BTMP7 | 0.50 | 0.15 |
| | BTMP8 | 1.03924 | 0.186638 | BTMP8 | 0.65 | 0.10 |
| | BTMP9 | 0.577613 | 0.104948 | BTMP9 | 0.67 | 0.12 |
| | BTMP10 | 0.673839 | 0.100383 | BTMP10 | 2.27 | 2.00 |
| foam cells | None | 0.89199 | 0.159893 | none | 0.79 | 0.09 |
| | BTMP1 | 0.912541 | 0 | BTMP1 | 2.19 | 0.46 |
| | BTMP2 | 0.763449 | 0.203646 | BTMP2 | 2.31 | 0.28 |
| | BTMP3 | 0.722072 | 0.2582 | BTMP3 | 1.66 | 0.50 |
| | BTMP4 | 1.081277 | 0.053871 | BTMP4 | 1.77 | 0.30 |
| | BTMP5 | 0.73216 | 0.14501 | BTMP5 | 2.48 | 0.01 |
| | BTMP6 | 1.445971 | 0 | BTMP6 | 2.15 | 0.53 |
| | BTMP7 | 0.690174 | 0 | BTMP7 | 1.15 | 0.07 |
| | BTMP8 | 1.013483 | 0.109235 | BTMP8 | 1.44 | 1.46 |
| | BTMP9 | 0.805138 | 0.046792 | BTMP9 | ND | |
| | BTMP10 | 0.556831 | 0.159653 | BTMP10 | 0.88 | 0.63 |

TABLE 5
Taqman results

| | IL-10 compound | | | IL-10 compound | | |
|---|---|---|---|---|---|---|
| | treatment 2 hours | | | treatment 8 hours | | |
| | | mean | s.d. | | mean | s.d. |
| macrophages | None | 0.838719 | 0.084748 | none | 0.96 | 0.23 |
| | BTMP1 | 0.93201 | 0.073727 | BTMP1 | 1.25 | 0.31 |
| | BTMP2 | 1.078281 | 0.162801 | BTMP2 | 1.97 | 0.17 |
| | BTMP3 | 0.949998 | 0.155105 | BTMP3 | 1.14 | 0.10 |
| | BTMP4 | 0.794645 | 0.120295 | BTMP4 | 1.28 | 0.23 |
| | BTMP5 | ND | | BTMP5 | 1.56 | 0.77 |
| | BTMP6 | 0.799543 | 0.059873 | BTMP6 | 0.83 | 0.20 |
| | BTMP7 | 0.95685 | 0.180639 | BTMP7 | 1.29 | 0.46 |
| | BTMP8 | 0.79994 | 0.060407 | BTMP8 | 1.39 | 0.25 |
| | BTMP9 | 0.623505 | 0.04854 | BTMP9 | 0.81 | 0.20 |
| | BTMP10 | 0.604754 | | BTMP10 | 1.91 | 0.80 |
| foam cells | None | 0.680602 | 0.118096 | none | 0.471046 | 0.074507 |
| | BTMP1 | 0.628717 | 0.031388 | BTMP1 | 1.34 | 0.19 |
| | BTMP2 | 0.788278 | 0.120264 | BTMP2 | 1.34 | 0.42 |
| | BTMP3 | 0.564875 | 0.022564 | BTMP3 | 0.91 | 0.10 |
| | BTMP4 | 0.724893 | 0.06027 | BTMP4 | 1.16 | 0.16 |
| | BTMP5 | 0.801344 | 0.047998 | BTMP5 | 0.99 | 0.05 |
| | BTMP6 | 0.936911 | 0.309266 | BTMP6 | 1.07 | 0.12 |
| | BTMP7 | 0.56192 | 0.028053 | BTMP7 | 1.01 | 0.17 |
| | BTMP8 | 0.745603 | 0.103929 | BTMP8 | 0.83 | 0.05 |
| | BTMP9 | 0.682842 | 0.090676 | BTMP9 | ND | |
| | BTMP10 | 0.518613 | 0.032787 | BTMP10 | 1.01 | 0.04 |

TABLE 6
Fold differences of selected chemokines as
determined by Affymetrix microarray

| | Effect of | Additional | Additional | Additional |
|---|---|---|---|---|
| | myelin | effect | effect | effect |
| Chemokine | ingestion | of BTMP1 | of BTMP2 | of BTMP9 |
| CCL2 | −1.6 | 3.27 | 5.3 | 3.1 |
| CCL3 | 2.5 | 1.1 | 1.1 | 1.0 |
| CCL4 | 3.3 | 1.3 | 1.4 | 1.1 |
| CCL5 | 4.3 | 1.4 | 2.1 | 1.7 |
| CCL7 | 1.6 | 1.6 | 2.5 | 1.9 |
| CXCL3 | 1.0 | 1.8 | 2.3 | 1.9 |
| CXCL8 | 5.1 | 1.5 | 2.0 | 1.8 |
| CCL18 | 3 | 2.8 | 4.5 | 3.3 |

### REFERENCES

Ares M. P., M. Stollenwerk, A. Olsson, B. Kallin, S. Jovinge, and J. Nilsson. Decreased inducibility of TNF expression in lipid-loaded macrophages. BMC Immunol. 2002; 3:13.
Autilio L. A., W. T. Norton, and R. D. Terry. The Preparation and Some Properties of Purified Myelin from the Central Nervous System. J. Neurochem. 1964; 11:17-27.
Becher B., A. Prat, and J. P. Antel. Brain-immune connection: immuno-regulatory properties of CNS-resident cells. Glia 2000; 29:293-304.
Cannella B. and C. S. Raine. Multiple sclerosis: Cytokine receptors on oligodendrocytes predict innate regulation. Ann. Neurol. 2004; 55:46-57.
Chayen J. and L. Bitensky. Analysis of chemical components of cells and tissues; reactions for lipids. Practical Histochemistry. West Sussex: Wiley, 1991:45.
Goerdt S., O. Politz, K. Schledzewski, R. Birk, A. Gratchev, and P. Guillot, et al. Alternative versus classical activation of macrophages. Pathobiology 1999; 67:222-6.
Gordon S. Alternative activation of macrophages. Nat. Rev. Immunol. 2003; 3:23-35.
Gratchev A., K. Schledzewski, P. Guillot, and S. Goerdt. Alternatively activated antigen-presenting cells: molecular repertoire, immune regulation, and healing. Skin Pharmacol. Appl. Skin Physiol. 2001; 14:272-9.
Greaves D. R. and S. Gordon. Thematic review series: The Immune System and Atherogenesis. Recent insights into the biology of macrophage scavenger receptors. J. Lipid Res. 2005; 46:11-20.
Greter M., F. L. Heppner, M. P. Lemos, B. M. Odermatt, N. Goebels, and T. Laufer, et al. Dendritic cells permit immune invasion of the CNS in an animal model of multiple sclerosis. Nat. Med. 2005; 11:328-34.
Grigoriadis N., T. Ben-Hur, Karussis, and I. Milonas. Axonal damage in multiple sclerosis: a complex issue in a complex disease. Clin. Neurol. Neurosurg. 2004; 106:211-7.
Harris S. G., J. Padilla, L. Koumas, D. Ray, and R. P. Phipps. Prostaglandins as modulators of immunity. Trends Immunol. 2002; 23:144-50.
Heppner F. L., M. Greter, D. Marino, J. Falsig, G. Raivich, and N. Hovelmeyer, et al. Experimental autoimmune encephalomyelitis repressed by microglial paralysis. Nat. Med. 2005; 11:146-52.
Hinz B., K. Brune, and A. Pahl. Prostaglandin E(2) up-regulates cyclooxygenase-2 expression in lipopolysaccharide-stimulated RAW 264.7 macrophages. Biochem. Biophys. Res. Commun. 2000; 272:744-8.
Hoefakker S., W. J. Boersma, and E. Claassen. Detection of human cytokines in situ using antibody and probe based methods. J. Immunol. Methods 1995; 185:149-75.
Ikegami R., Y. Sugimoto, E. Segi, M. Katsuyama, H. Karahashi, F. Amano, et al. The expression of prostaglandin E receptors EP2 and EP4 and their different regulation by lipopolysaccharide in C3H/HeN peritoneal macrophages. J. Immunol. 2001; 166:4689-96.
Joseph S. B., M. N. Bradley, A. Castrillo, K. W. Bruhn, P. A. Mak, L Pei, et al. LXR-dependent gene expression is important for macrophage survival and the innate immune response. Cell 2004; 119:299-309.
Joseph S. B., A. Castrillo, B. A. Laffitte, D. J. Mangelsdorf, and P. Tontonoz. Reciprocal regulation of inflammation and lipid metabolism by liver X receptors. Nat. Med. 2003; 9:213-9.
Kalinski P., C. M. Hilkens, A. Snijders, F. G. Snijdewint, and M. L. Kapsenberg. Dendritic cells, obtained from peripheral blood precursors in the presence of PGE2, promote Th2 responses. Adv. Exp. Med. Biol. 1997; 417:363-7.
Kodelja V., C. Muller, O. Politz, N. Hakij, C. E. Orfanos, and S. Goerdt. Alternative macrophage activation-associated CC-chemokine-1, a novel structural homologue of macrophage inflammatory protein-1 alpha with a Th2-associated expression pattern. J. Immunol. 1998; 160:1411-8.
Kuhlmann T., G. Lingfeld, A. Bitsch, J. Schuchardt, and W. Bruck. Acute axonal damage in multiple sclerosis is most extensive in early disease stages and decreases over time. Brain 2002; 125:2202-12.
Lassmann H. Recent neuropathological findings in MS—implications for diagnosis and therapy. J. Neurol. 2004; 251 Suppl 4:IV2-5.
Lawrence T., D. A. Willoughby, and D. W. Gilroy. Anti-inflammatory lipid mediators and insights into the resolution of inflammation. Nat. Rev. Immunol. 2002; 2:787-95.
Li H., M. L. Cuzner, and J. Newcombe. Microglia-derived macrophages in early multiple sclerosis plaques. Neuropathol. Appl. Neurobiol. 1996; 22:207-15.
Lo C. J., M. Fu, F. R. Lo, and H. G. Cryer. Macrophage TNF mRNA expression induced by LPS is regulated by sphingomyelin metabolites. Shock 1999; 11:411-5.
Mantovani A., A. Sica, S. Sozzani, P. Allavena, A. Vecchi, and M. Locati. The chemokine system in diverse forms of macrophage activation and polarization. Trends Immunol. 2004; 25:677-86.
Mantovani A., S. Sozzani, M. Locati, P. Allavena, and A. Sica. Macrophage polarization: tumor-associated macrophages as a paradigm for polarized M2 mononuclear phagocytes. Trends Immunol. 2002; 23:549-55.
Matute C. and F. Perez-Cerda. Multiple sclerosis: novel perspectives on newly forming lesions. Trends Neurosci. 2005; 28:173-5.
McMahon E. J., S. L. Bailey, C. V. Castenada, H. Waldner and S. D. Miller. Epitope spreading initiates in the CNS in two mouse models of multiple sclerosis. Nat. Med. 2005; 11:335-339.
Mosser D. M. The many faces of macrophage activation. J. Leukoc. Biol. 2003; 73:209-12.
Newcombe J., H. Li, and M. L. Cuzner. Low density lipoprotein uptake by macrophages in multiple sclerosis plaques: implications for pathogenesis. Neuropathol. Appl. Neurobiol. 1994; 20:152-62.
Norton W. T. and S. E. Podusio. Myelination in rat brain: method of myelin isolation. J. Neurochem. 1973; 21:749-57.
Pettus B. J., C. E. Chalfant, and Y. A. Hannun. Ceramide in apoptosis: an overview and current perspectives. Biochim. Biophys. Acta. 2002; 1585:114-25.
Platten M. and L. Steinman. Multiple sclerosis: trapped in deadly glue. Nat. Med. 2005; 11:252-3.
Raine C. S. Multiple sclerosis: immune system molecule expression in the central nervous system. J. Neuropathol. Exp. Neurol. 1994; 53:328-37.
Sospedra M. and R. Martin. Immunology of multiple sclerosis *. Annu. Rev. Immunol. 2005; 23:683-747
Van der Fits L., L. I. van der Wel, J. D. Laman, E. P. Prens, and M. C. Verschuren. Psoriatic lesional skin exhibits an aberrant expression pattern of interferon regulatory factor-2 (IRF-2). J. Pathol. 2003; 199:107-14.
Van der Laan L. J., S. R. Ruuls, K. S. Weber, I. J. Lodder, E. A. Dopp, and C. D. Dijkstra. Macrophage phagocytosis of myelin in vitro determined by flow cytometry: phagocytosis is mediated by CR3 and induces production of tumor necrosis factor-alpha and nitric oxide. J. Neuroimmunol. 1996; 70:145-52.
Varadhachary A. S., M. Monestier, and P. Salgame. Reciprocal induction of IL-10 and IL-12 from macrophages by low-density lipoprotein and its oxidized forms. Cell Immunol. 2001; 213:45-51
Vulcano M., S. Struyf, P. Scapini, M. Cassatella, S. Bernasconi, and R. Bonecchi, et al. Unique regulation of CCL18 production by maturing dendritic cells. J. Immunol. 2003; 170:3843-9.
Wingerchuk D. M., C. F. Lucchinetti, and J. H. Noseworthy. Multiple sclerosis: current pathophysiological concepts. Lab. Invest. 2001; 81:263-81.
Woelk H. and P. Borri. Lipid and fatty acid composition of myelin purified from normal and MS brains. Eur. Neurol. 1973; 10:250-60.

1. A method for assessing activity of a compound comprising
a. culturing cells;
b. contacting at least one of said cultured cells with a lipid-rich fraction;
c. contacting at least one of said cultured cells with said test compound;
d. determining the presence of a gene product of at least one cell of said cultured cells; and
e. optionally determining the presence of said gene product of at least one cultured cell not contacted with said test compound.

2. A method according to claim 1, wherein said cell is a myeloid cell.

3. A method according to claim 1, wherein said cell is a peripheral blood cell taken from a subject.

4. A method according to claim 1, wherein said cell has been derived from a subject thought to be suffering from a disease.

5. A method according to claim 4, wherein foamy cells are considered involved in the etiology of said disease.

6. A method according to claim 5, wherein said disease is Multiple Sclerosis.

7. A method according to claim 1, wherein a myeloid cell line such as U937 (human): ATCC CRL-1593.2; THP-1 (human): ATCC TIB-202; RAW (mouse): ATCC TIB-71 is used.

8. A method according to claim 1, wherein said lipid-rich fraction comprises phospholipid.

9. A method according to claim 1, wherein said lipid-rich fraction comprises myelin.

10. A method according to claim 1, wherein said gene product is a proteinaceous substance.

11. A method according to claim 10, wherein said gene product is a cytokine or chemokine.

12. A method according to claim 1, wherein said gene product is RNA.

13. A method according to claim 12, wherein said RNA at least partially encodes a cytokine or chemokine.

14. A method according to claim 1, wherein said cell is of human origin.
